SEQUENCE LISTING (177 sequences)

SEQ ID NO: 1 - protein, 5 aa, Artificial Sequence
  anti-oxMIF
IYTMD

SEQ ID NO: 2 - protein, 17 aa, Artificial Sequence
  anti-oxMIF
YISPSGGNTSYADSVKG

SEQ ID NO: 3 - protein, 17 aa, Artificial Sequence
  anti-oxMIF
RQYVLRYFDWSADAFDI

SEQ ID NO: 4 - protein, 11 aa, Artificial Sequence
  anti-oxMIF
RASQSISSYLN

SEQ ID NO: 5 - protein, 7 aa, Artificial Sequence
  anti-oxMIF
AASSLQS

SEQ ID NO: 6 - protein, 9 aa, Artificial Sequence
  anti-oxMIF
QQSYSTPWT

SEQ ID NO: 7 - protein, 5 aa, Artificial Sequence
  anti-oxMIF
IYSMN

SEQ ID NO: 8 - protein, 17 aa, Artificial Sequence
  anti-oxMIF
SIGSSGGTTYYADSVKG

SEQ ID NO: 9 - protein, 9 aa, Artificial Sequence
  anti-oxMIF
SQWLYGMDV

SEQ ID NO: 10 - protein, 11 aa, Artificial Sequence
  anti-oxMIF
RSSQRIMTYLN

SEQ ID NO: 11 - protein, 7 aa, Artificial Sequence
  anti-oxMIF
VASHSQS

SEQ ID NO: 12 - protein, 9 aa, Artificial Sequence
  anti-oxMIF
QQSFWTPLT

SEQ ID NO: 13 - protein, 5 aa, Artificial Sequence
  anti-oxMIF
KYYMI

SEQ ID NO: 14 - protein, 17 aa, Artificial Sequence
  anti-oxMIF
WIGPSGGFTFYADSVKG

SEQ ID NO: 15 - protein, 12 aa, Artificial Sequence
  anti-oxMIF
GTPDYGGNSLDH

SEQ ID NO: 16 - protein, 11 aa, Artificial Sequence
  anti-oxMIF
RASQSIGTYLS

SEQ ID NO: 17 - protein, 7 aa, Artificial Sequence
  anti-oxMIF
ATSRLQS

SEQ ID NO: 18 - protein, 9 aa, Artificial Sequence
  anti-oxMIF
QQTYSTPLT

SEQ ID NO: 19 - protein, 5 aa, Artificial Sequence
  anti-oxMIF
IYAMD

SEQ ID NO: 20 - protein, 17 aa, Artificial Sequence
  anti-oxMIF
GIVPSGGFTKYADSVKG

SEQ ID NO: 21 - protein, 18 aa, Artificial Sequence
  anti-oxMIF
VNVIAVAGTGYYYYGMDV

SEQ ID NO: 22 - protein, 12 aa, Artificial Sequence
  anti-oxMIF
RASQGVSSSSLA

SEQ ID NO: 23 - protein, 7 aa, Artificial Sequence
  anti-oxMIF
GTSSRAT

SEQ ID NO: 24 - protein, 8 aa, Artificial Sequence
  anti-oxMIF
QQYGRSLT

SEQ ID NO: 25 - protein, 7 aa, Artificial Sequence
  anti-oxMIF
GASNRAT

SEQ ID NO: 26 - protein, 5 aa, Artificial Sequence
  anti-oxMIF
WYAMD

SEQ ID NO: 27 - protein, 17 aa, Artificial Sequence
  anti-oxMIF
GIYPSGGRTKYADSVKG

SEQ ID NO: 28 - protein, 12 aa, Artificial Sequence
  anti-oxMIF
RASQGVSSSSLA

SEQ ID NO: 29 - protein, 30 aa, Artificial Sequence
  anti-oxMIF
EVQLLESGGGLVQPGGSLRLSCAASGFTFS

SEQ ID NO: 30 - protein, 14 aa, Artificial Sequence
  anti-oxMIF
WVRQAPGKGLEWVS

SEQ ID NO: 31 - protein, 32 aa, Artificial Sequence
  anti-oxMIF
RFTISRDNSKNTLYLQMNSLRAEDTAVYYCAS

SEQ ID NO: 32 - protein, 11 aa, Artificial Sequence
  anti-oxMIF
WGQGTMVTVSS

SEQ ID NO: 33 - protein, 23 aa, Artificial Sequence
  anti-oxMIF
DIQMTQSPSSLSASVGDRVTITC

SEQ ID NO: 34 - protein, 15 aa, Artificial Sequence
  anti-oxMIF
WYQQKPGKAPKLLIY

SEQ ID NO: 35 - protein, 32 aa, Artificial Sequence
  anti-oxMIF
GVPSRFSGSGSGTDFTLTISSLQPEDFATYYC

SEQ ID NO: 36 - protein, 10 aa, Artificial Sequence
  anti-oxMIF
FGQGTKVEIK

SEQ ID NO: 37 - protein, 30 aa, Artificial Sequence; identical to SEQ ID NO: 29
  anti-oxMIF

SEQ ID NO: 38 - protein, 14 aa, Artificial Sequence
  anti-oxMIF
WVRQAPGKGLEWVS

SEQ ID NO: 39 - protein, 32 aa, Artificial Sequence
  anti-oxMIF
RFTISRDNSKNTLYLQMNSLRAEDTAVYYCAG

SEQ ID NO: 40 - protein, 11 aa, Artificial Sequence
  anti-oxMIF
WGQGTTVTVSS

SEQ ID NO: 41 - protein, 23 aa, Artificial Sequence
  anti-oxMIF
DIQMTQSPSSLSASVGDRVTITC

SEQ ID NO: 42 - protein, 15 aa, Artificial Sequence
  anti-oxMIF
WYQQKPGKAPKLLIF

SEQ ID NO: 43 - protein, 32 aa, Artificial Sequence
  anti-oxMIF
GVPSRFRGSGSETDFTLTISGLQPEDSATYYC

SEQ ID NO: 44 - protein, 10 aa, Artificial Sequence
  anti-oxMIF
FGGGTKVEIK

SEQ ID NO: 45 - protein, 30 aa, Artificial Sequence; identical to SEQ ID NO: 29
  anti-oxMIF

SEQ ID NO: 46 - protein, 14 aa, Artificial Sequence
  anti-oxMIF
WVRQAPGKGLEWVS

SEQ ID NO: 47 - protein, 32 aa, Artificial Sequence
  anti-oxMIF
RFTISRDNSKNTLYLQMNSLRAEDTAVYYCAR

SEQ ID NO: 48 - protein, 11 aa, Artificial Sequence
  anti-oxMIF
WGQGTLVTVSS

SEQ ID NO: 49 - protein, 23 aa, Artificial Sequence
  anti-oxMIF
DIQMTQSPSSLPASVGDRVTITC

SEQ ID NO: 50 - protein, 15 aa, Artificial Sequence
  anti-oxMIF
WYQHKPGNAPKLLIY

SEQ ID NO: 51 - protein, 32 aa, Artificial Sequence
  anti-oxMIF
GVPSRFSGGGSGTRFTLAISSLQPDDFATYFC

SEQ ID NO: 52 - protein, 10 aa, Artificial Sequence
  anti-oxMIF
FGGGTKVDIK

SEQ ID NO: 53 - protein, 30 aa, Artificial Sequence; identical to SEQ ID NO: 29
  anti-oxMIF

SEQ ID NO: 54 - protein, 14 aa, Artificial Sequence
  anti-oxMIF
WVRQAPGKGLEWVS

SEQ ID NO: 55 - protein, 32 aa, Artificial Sequence; identical to SEQ ID NO: 47
  anti-oxMIF

SEQ ID NO: 56 - protein, 11 aa, Artificial Sequence
  anti-oxMIF
WGQGTTVTVSS

SEQ ID NO: 57 - protein, 23 aa, Artificial Sequence
  anti-oxMIF
DIQMTQSPGTLSLSPGERATLSC

SEQ ID NO: 58 - protein, 15 aa, Artificial Sequence
  anti-oxMIF
WYQQKPGQAPRLLIY

SEQ ID NO: 59 - protein, 32 aa, Artificial Sequence
  anti-oxMIF
GIPDRFSGSASGTDFTLTISRLQPEDFAVYYC

SEQ ID NO: 60 - protein, 10 aa, Artificial Sequence
  anti-oxMIF
FGGGTKVEIK

SEQ ID NO: 61 - protein, 30 aa, Artificial Sequence; identical to SEQ ID NO: 29
  anti-oxMIF

SEQ ID NO: 62 - protein, 14 aa, Artificial Sequence
  anti-oxMIF
WVRQAPGKGLEWVS

SEQ ID NO: 63 - protein, 32 aa, Artificial Sequence; identical to SEQ ID NO: 47
  anti-oxMIF

SEQ ID NO: 64 - protein, 11 aa, Artificial Sequence
  anti-oxMIF
WGQGTTVTVSS

SEQ ID NO: 65 - protein, 23 aa, Artificial Sequence
  anti-oxMIF
DIQMTQSPVTLSLSPGERATLSC

SEQ ID NO: 66 - protein, 15 aa, Artificial Sequence
  anti-oxMIF
WYQQKPGQTPRLLIY

SEQ ID NO: 67 - protein, 32 aa, Artificial Sequence
  anti-oxMIF
GIPDRFSGSGSGTDFTLTISRLEPEDFAVYYC

SEQ ID NO: 68 - protein, 10 aa, Artificial Sequence
  anti-oxMIF
FGGGTKVEIK

SEQ ID NO: 69 - protein, 30 aa, Artificial Sequence; identical to SEQ ID NO: 29
  anti-oxMIF

SEQ ID NO: 70 - protein, 14 aa, Artificial Sequence
  anti-oxMIF
WVRQAPGKGLEWVS

SEQ ID NO: 71 - protein, 32 aa, Artificial Sequence; identical to SEQ ID NO: 47
  anti-oxMIF

SEQ ID NO: 72 - protein, 11 aa, Artificial Sequence
  anti-oxMIF
WGQGTTVTVSS

SEQ ID NO: 73 - protein, 23 aa, Artificial Sequence
  anti-oxMIF
DIQMTQSPGTLSLSPGERATLSC

SEQ ID NO: 74 - protein, 15 aa, Artificial Sequence
  anti-oxMIF
WYQQKPGQAPRLLIY

SEQ ID NO: 75 - protein, 32 aa, Artificial Sequence; identical to SEQ ID NO: 59
  anti-oxMIF

SEQ ID NO: 76 - protein, 10 aa, Artificial Sequence
  anti-oxMIF
FGGGTKVEIK

SEQ ID NO: 77 - protein, 5 aa, Artificial Sequence
  anti-CD3 heavy chain sequence
RYTMH

SEQ ID NO: 78 - protein, 17 aa, Artificial Sequence
  anti-CD3 heavy chain sequence
YINPSRGYTNYNQKFKD

SEQ ID NO: 79 - protein, 10 aa, Artificial Sequence
  anti-CD3 heavy chain sequence
YYDDHYCLDY

SEQ ID NO: 80 - protein, 10 aa, Artificial Sequence
  anti-CD3 light chain sequence
RASSSVSYMN

SEQ ID NO: 81 - protein, 7 aa, Artificial Sequence
  anti-CD3 light chain sequence
DTSKVAS

SEQ ID NO: 82 - protein, 9 aa, Artificial Sequence
  anti-CD3 light chain sequence
QQWSSNPLT

SEQ ID NO: 83 - protein, 10 aa, Artificial Sequence
  anti-CD3 light chain sequence
SASSSVSYMN

SEQ ID NO: 84 - protein, 7 aa, Artificial Sequence
  anti-CD3 light chain sequence
DTSKLAS

SEQ ID NO: 85 - protein, 9 aa, Artificial Sequence
  anti-CD3 light chain sequence
QQWSSNPFT

SEQ ID NO: 86 - protein, 5 aa, Artificial Sequence
  anti-CD3 heavy chain sequence
GYGMH

SEQ ID NO: 87 - protein, 17 aa, Artificial Sequence
  anti-CD3 heavy chain sequence
VIWYDGSKKYYVDSVKG

SEQ ID NO: 88 - protein, 9 aa, Artificial Sequence
  anti-CD3 heavy chain sequence
QMGYWHFDL

SEQ ID NO: 89 - protein, 11 aa, Artificial Sequence
  anti-CD3 light chain sequence
RASQSVSSYLA

SEQ ID NO: 90 - protein, 7 aa, Artificial Sequence
  anti-CD3 light chain sequence
DASNRAT

SEQ ID NO: 91 - protein, 10 aa, Artificial Sequence
  anti-CD3 light chain sequence
QQRSNWPPLT

SEQ ID NO: 92 - protein, 5 aa, Artificial Sequence
  anti-CD3 heavy chain sequence
SFPMA

SEQ ID NO: 93 - protein, 17 aa, Artificial Sequence
  anti-CD3 heavy chain sequence
TISTSGGRTYYRDSVKG

SEQ ID NO: 94 - protein, 10 aa, Artificial Sequence
  anti-CD3 heavy chain sequence
FRQYSGGFDY

SEQ ID NO: 95 - protein, 13 aa, Artificial Sequence
  anti-CD3 light chain sequence
TLSSGNIENNYVH

SEQ ID NO: 96 - protein, 7 aa, Artificial Sequence
  anti-CD3 light chain sequence
DDDKRPD

SEQ ID NO: 97 - protein, 9 aa, Artificial Sequence
  anti-CD3 light chain sequence
HSYVSSFNV

SEQ ID NO: 98 - protein, 30 aa, Artificial Sequence
  anti-CD3 heavy chain sequence
DIKLQQSGAELARPGASVKMSCKTSGYTFT

SEQ ID NO: 99 - protein, 14 aa, Artificial Sequence
  anti-CD3 heavy chain sequence
WVKQRPGQGLEWIG

SEQ ID NO: 100 - protein, 32 aa, Artificial Sequence
  anti-CD3 heavy chain sequence
KATLTTDKSSSTAYMQLSSLTSEDSAVYYCAR

SEQ ID NO: 101 - protein, 11 aa, Artificial Sequence
  anti-CD3 heavy chain sequence
WGQGTTLTVSS

SEQ ID NO: 102 - protein, 23 aa, Artificial Sequence
  anti-CD3 light chain sequence
DIQLTQSPAIMSASPGEKVTMTC

SEQ ID NO: 103 - protein, 15 aa, Artificial Sequence
  anti-CD3 light chain sequence
WYQQKSGTSPKRWIY

SEQ ID NO: 104 - protein, 32 aa, Artificial Sequence
  anti-CD3 light chain sequence
GVPYRFSGSGSGTSYSLTISSMEAEDAATYYC

SEQ ID NO: 105 - protein, 10 aa, Artificial Sequence
  anti-CD3 light chain sequence
FGAGTKLELK

SEQ ID NO: 106 - protein, 30 aa, Artificial Sequence
  anti-CD3 heavy chain sequence
QVQLQQSGAELARPGASVKMSCKASGYTFT

SEQ ID NO: 107 - protein, 23 aa, Artificial Sequence
  anti-CD3 light chain sequence
QIVLTQSPAIMSASPGEKVTMTC

SEQ ID NO: 108 - protein, 32 aa, Artificial Sequence
  anti-CD3 light chain sequence
GVPAHFRGSGSGTSYSLTISGMEAEDAATYYC

SEQ ID NO: 109 - protein, 10 aa, Artificial Sequence
  anti-CD3 light chain sequence
FGSGTKLEIN

SEQ ID NO: 110 - protein, 30 aa, Artificial Sequence
  anti-CD3 heavy chain sequence
QVQLVQSGGGVVQPGRSLRLSCKASGYTFT

SEQ ID NO: 111 - protein, 14 aa, Artificial Sequence
  anti-CD3 heavy chain sequence
WVRQAPGKGLEWIG

SEQ ID NO: 112 - protein, 32 aa, Artificial Sequence
  anti-CD3 heavy chain sequence
RFTISRDNSKNTAFLQMDSLRPEDTGVYFCAR

SEQ ID NO: 113 - protein, 11 aa, Artificial Sequence
  anti-CD3 heavy chain sequence
WGQGTPVTVSS

SEQ ID NO: 114 - protein, 23 aa, Artificial Sequence
  anti-CD3 light chain sequence
DIQMTQSPSSLSASVGDRVTITC

SEQ ID NO: 115 - protein, 15 aa, Artificial Sequence
  anti-CD3 light chain sequence
WYQQTPGKAPKRWIY

SEQ ID NO: 116 - protein, 32 aa, Artificial Sequence
  anti-CD3 light chain sequence
GVPSRFSGSGSGTDYTFTISSLQPEDIATYYC

SEQ ID NO: 117 - protein, 10 aa, Artificial Sequence
  anti-CD3 light chain sequence
FGQGTKLQIT

SEQ ID NO: 118 - protein, 30 aa, Artificial Sequence
  anti-CD3 heavy chain sequence
QVQLVESGGGVVQPGRSLRLSCAASGFKFS

SEQ ID NO: 119 - protein, 14 aa, Artificial Sequence
  anti-CD3 heavy chain sequence
WVRQAPGKGLEWVA

SEQ ID NO: 120 - protein, 32 aa, Artificial Sequence; identical to SEQ ID NO: 47
  anti-CD3 heavy chain sequence

SEQ ID NO: 121 - protein, 11 aa, Artificial Sequence
  anti-CD3 heavy chain sequence
WGRGTLVTVSS

SEQ ID NO: 122 - protein, 23 aa, Artificial Sequence
  anti-CD3 light chain sequence
EIVLTQSPATLSLSPGERATLSC

SEQ ID NO: 123 - protein, 15 aa, Artificial Sequence
  anti-CD3 light chain sequence
WYQQKPGQAPRLLIY

SEQ ID NO: 124 - protein, 32 aa, Artificial Sequence
  anti-CD3 light chain sequence
GIPARFSGSGSGTDFTLTISSLEPEDFAVYYC

SEQ ID NO: 125 - protein, 10 aa, Artificial Sequence
  anti-CD3 light chain sequence
FGGGTKVEIK

SEQ ID NO: 126 - protein, 30 aa, Artificial Sequence; identical to SEQ ID NO: 29
  anti-CD3 heavy chain sequence

SEQ ID NO: 127 - protein, 14 aa, Artificial Sequence
  anti-CD3 heavy chain sequence
WVRQAPGKGLEWVS

SEQ ID NO: 128 - protein, 32 aa, Artificial Sequence
  anti-CD3 heavy chain sequence
RFTISRDNSKNTLYLQMNSLRAEDTAVYYCAK

SEQ ID NO: 129 - protein, 11 aa, Artificial Sequence
  anti-CD3 heavy chain sequence
WGQGTLVTVSS

SEQ ID NO: 130 - protein, 22 aa, Artificial Sequence
  anti-CD3 light chain sequence
DIQLTQPNSVSTSLGSTVKLSC

SEQ ID NO: 131 - protein, 15 aa, Artificial Sequence
  anti-CD3 light chain sequence
WYQLYEGRSPTTMIY

SEQ ID NO: 132 - protein, 34 aa, Artificial Sequence
  anti-CD3 light chain sequence
GVPDRFSGSIDRSSNSAFLTIHNVAIEDEAIYFC

SEQ ID NO: 133 - protein, 10 aa, Artificial Sequence
  anti-CD3 light chain sequence
FGGGTKLTVL

SEQ ID NO: 134 - protein, 107 aa, Artificial Sequence
  anti-oxMIF
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIK

SEQ ID NO: 135 - protein, 119 aa, Artificial Sequence
  anti-CD3 heavy chain sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 136 - protein, 106 aa, Artificial Sequence
  anti-CD3 light chain sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 137 - protein, 494 aa, Artificial Sequence
  anti-oxMIF
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGGGSGGGGSGGGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 138 - protein, 474 aa, Artificial Sequence
  anti-oxMIF
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGSGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGSGGSDIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSHHHHHH

SEQ ID NO: 139 - protein, 705 aa, Artificial Sequence
  anti-oxMIF
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 140 - protein, 214 aa, Artificial Sequence
  anti-oxMIF
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 207 aa, Artificial Sequence
  CD3 antibody
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 142 - protein, 171 aa, Artificial Sequence
  CD3 antibody
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 143 - protein, 182 aa, Artificial Sequence
  CD3 antibody
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 144 - protein, 115 aa, Artificial Sequence
  anti-oxMIF
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 145 - protein, 7 aa, Artificial Sequence
  anti-oxMIF
EPCALCS

SEQ ID NO: 146 - protein, 30 aa, Artificial Sequence
  anti-CD3 heavy chain sequence
QVQLVQSGAEVKKPGASVKVSCKASGYTFT

SEQ ID NO: 147 - protein, 14 aa, Artificial Sequence
  anti-CD3 heavy chain sequence
WVRQAPGQGLEWMG

SEQ ID NO: 148 - protein, 32 aa, Artificial Sequence
  anti-CD3 heavy chain sequence
RVTLTTDKSSSTAYMELSSLRSEDTAVYYCAR

SEQ ID NO: 149 - protein, 10 aa, Artificial Sequence
  anti-CD3 heavy chain sequence
YYDDHYSLDY

SEQ ID NO: 150 - protein, 15 aa, Artificial Sequence
  anti-CD3 light chain sequence
WYQQKPGKAPKRLIY

SEQ ID NO: 151 - protein, 7 aa, Artificial Sequence
  anti-CD3 light chain sequence
QQWSSNP

SEQ ID NO: 152 - protein, 12 aa, Artificial Sequence
  anti-CD3 light chain sequence
FTFGQGTKLEIK

SEQ ID NO: 153 - protein, 497 aa, Artificial Sequence
  anti-oxMIF / anti-CD3
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKAAAEQKLISEEDLAAHHHHHH

SEQ ID NO: 154 - protein, 214 aa, Artificial Sequence; identical to SEQ ID NO: 140
  anti-oxMIF / anti-CD3

SEQ ID NO: 155 - protein, 497 aa, Artificial Sequence; identical to SEQ ID NO: 153
  anti-oxMIF / anti-CD3

SEQ ID NO: 156 - protein, 477 aa, Artificial Sequence
  anti-oxMIF / anti-CD3
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGSGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKASAWSHPQFEK

SEQ ID NO: 157 - protein, 214 aa, Artificial Sequence; identical to SEQ ID NO: 140
  anti-oxMIF / anti-CD3

SEQ ID NO: 158 - protein, 448 aa, Artificial Sequence
  anti-oxMIF / anti-CD3
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 472 aa, Artificial Sequence
  anti-oxMIF / anti-CD3
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 160 - protein, 447 aa, Artificial Sequence
  anti-oxMIF / anti-CD3
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 469 aa, Artificial Sequence
  anti-oxMIF / anti-CD3
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 162 - protein, 214 aa, Artificial Sequence; identical to SEQ ID NO: 140
  anti-oxMIF / anti-CD3

SEQ ID NO: 163 - protein, 448 aa, Artificial Sequence; identical to SEQ ID NO: 158
  anti-oxMIF / anti-CD3

SEQ ID NO: 164 - protein, 453 aa, Artificial Sequence
  anti-oxMIF / anti-CD3
QVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 165 - protein, 211 aa, Artificial Sequence
  anti-oxMIF / anti-CD3
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 166 - protein, 703 aa, Artificial Sequence
  anti-oxMIF / anti-CD3
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 167 - protein, 214 aa, Artificial Sequence; identical to SEQ ID NO: 140
  anti-oxMIF / anti-CD3

SEQ ID NO: 168 - protein, 508 aa, Artificial Sequence
  anti-oxMIF / anti-CD3
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGGGSGGGGSGGGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKAAAEQKLISEEDLSAWSHPQFEK

SEQ ID NO: 169 - protein, 491 aa, Artificial Sequence
  anti-oxMIF / anti-CD3
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKGGSGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGSGGSDIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGSGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSAAAEQKLISEEDLSAWSHPQFEK

SEQ ID NO: 170 - protein, 447 aa, Artificial Sequence; identical to SEQ ID NO: 160
  anti-oxMIF / anti-CD3

SEQ ID NO: 171 - protein, 214 aa, Artificial Sequence; identical to SEQ ID NO: 140
  anti-oxMIF / anti-CD3

SEQ ID NO: 172 - protein, 118 aa, Artificial Sequence
  anti-oxMIF heavy chain sequence
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSS

SEQ ID NO: 173 - protein, 476 aa, Artificial Sequence
  anti-oxMIF / anti-CD3
EVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 174 - protein, 476 aa, Artificial Sequence; identical to SEQ ID NO: 173
  anti-oxMIF / anti-CD3

SEQ ID NO: 175 - protein, 466 aa, Artificial Sequence
  anti-oxMIF / anti-CD3
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGSGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 176 - protein, 485 aa, Artificial Sequence
  anti-oxMIF / anti-CD3
DIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGGGSGGGGSGGGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSSGGSGGSGGSGGSGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIK

SEQ ID NO: 177 - protein, 468 aa, Artificial Sequence
  anti-oxMIF / anti-CD3
DIQMTQSPSSLSASVGDRVTITCSASSSVSYMNWYQQKPGKAPKRLIYDTSKLASGVPSRFSGSGSGTDFTLTISSLQPEDFATYYCQQWSSNPFTFGQGTKLEIKGGSGGSEVQLLESGGGLVQPGGSLRLSCAASGFTFSIYSMNWVRQAPGKGLEWVSSIGSSGGTTYYADSVKGRFTISRDNSKNTLYLQMNSLRAEDTAVYYCAGSQWLYGMDVWGQGTTVTVSSGGSGGSDIQMTQSPSSLSASVGDRVTITCRSSQRIMTYLNWYQQKPGKAPKLLIFVASHSQSGVPSRFRGSGSETDFTLTISGLQPEDSATYYCQQSFWTPLTFGGGTKVEIKGGSGGSQVQLVQSGAEVKKPGASVKVSCKASGYTFTRYTMHWVRQAPGQGLEWMGYINPSRGYTNYNQKFKDRVTLTTDKSSSTAYMELSSLRSEDTAVYYCARYYDDHYSLDYWGQGTLVTVSS

(19) United States
(12) Patent Application Publication
(10) Pub. No.: US 2022/0002398 A1
(43) Pub. Date: Jan. 6, 2022

(54) ANTI-oxMIF/ANTI-CD3 ANTIBODY FOR CANCER TREATMENT

(71) Applicant: OncoOne Research & Development GmbH, Klostemeuburg (AT)

(72) Inventors: Michael Robert THIELE, Vienna (AT); Alexander SCHINAGL, Vienna (AT); Randolf KERSCHBAUMER, Klosterneuburg (AT)

(21) Appl. No.: 17/055,092

(22) PCT Filed: Jun. 7, 2019
(86) PCT No.: PCT/EP2019/065023

§ 371 Date: Nov. 12, 2020

(30) Foreign Application Priority Data
Jun. 7, 2018 (EP) 18176612.2

(51) Int. Cl. C07K 16/24; C07K 16/28; A61P 35/00

(52) U.S. Cl. CPC C07K 16/24; C07K 16/2809; A61P 35/00; C07K 2317/31; C07K 2317/92; C07K 2317/35; C07K 2317/55; C07K 2317/622; C07K 2317/565

(57) ABSTRACT

The invention refers to an anti-ox MIF/anti-CD3 antibody comprising at least one binding site specifically recognizing ox MIF and at least one binding site specifically recognizing CD3 and its use in the treatment of hyperproliferative diseases, specifically in the treatment of cancers.

### CROSS-REFERENCE TO RELATED APPLICATIONS

This application is the U.S. national stage of International Patent Application No. PCT/EP2019/065023, filed on Jun. 7, 2019 and entitled ANTI-oxMIF/ANTI-CD3 ANTIBODY FOR CANCER TREATMENT, which claims the benefit of priority under 35 U.S.C. § 119 from European Patent Application No. 18176612.2, filed Jun. 7, 2018. The disclosures of the foregoing applications are incorporated herein by reference in their entirety.

### SEQUENCE LISTING

The entire content of a Sequence Listing titled “Sequence_Listing.txt,” created on Jul. 23, 2021 and having a size of 149 kilobytes, which has been submitted in electronic form in connection with the present application, is hereby incorporated by reference herein in its entirety.

### FIELD OF THE INVENTION

The invention refers to an anti-oxMIF/anti-CD3 antibody comprising at least one binding site specifically recognizing oxMIF and at least one binding site specifically recognizing CD3 and its use in the treatment of hyperproliferative diseases, specifically in the treatment of cancer.

### BACKGROUND

The cytokine Macrophage Migration Inhibitory Factor (MIF) has been described as early as 1966 (David, J. R., 1966, Proc. Natl. Acad. Sci. U.S.A. 56, 72-77; Bloom B. R. and Bennet, B., 1966, Science 153, 80-82). MIF, however, is markedly different from other cytokines and chemokines because it is constitutively expressed, stored in the cytoplasm and present in the circulation of healthy subjects. Due to the ubiquitous nature of this protein, MIF can be considered as an inappropriate target for therapeutic intervention. However, MIF occurs in two immunologically distinct conformational isoforms, termed reduced MIF (redMIF) and oxidized MIF (oxMIF) (Thiele M. et al., J Immunol 2015; 195:2343-2352). RedMIF was found to be the abundantly expressed isoform of MIF that can be found in the cytoplasm and in the circulation of any subject. RedMIF seems to represent a latent non-active storage form (Schinagl. A. et al., Biochemistry. 2018 Mar. 6; 57(9):1523-1532).

In contrast, oxMIF seems to be the physiologic relevant and disease related isoform which can be detected specifically in tumor tissue from patients with colorectal, pancreatic, ovarian and lung cancer (Schinagl. A. et al., Oncotarget. 2016 Nov. 8; 7(45):73486-73496).

The number of successful drug targets to treat cancers, like the above mentioned oxMIF positive indications, is restricted. E.g. more than 300 potential immune-oncology targets are described, but many clinical studies focus on anti-PD1 and anti-PDL1 antibodies (Tang J., et al. Ann Oncol. 2018 Jan. 1; 29(1):84-91). The scientific and medical community therefore eagerly awaits potential drugs targeting tumor specific antigens to increase the therapeutic options for cancer patients with poor prognosis.

OxMIF seems to be highly tumor specific, and antibodies targeting oxMIF show efficacy in vitro and in animal studies (Hussain F. et al., Mol Cancer Ther. 2013 July; 12(7):1223-34; Schinagl. A. et al., Oncotarget. 2016 Nov. 8; 7(45):73486-73496). An oxMIF specific antibody demonstrated an acceptable safety profile, satisfactory tissue penetration and indications for anti-tumor activity in a phase 1 clinical trial (Mahalingam D. et al., 2015, ASCO Abstract ID2518). However, the mode of action of anti-oxMIF antibodies seems to be solely based on neutralization of the biologic activity of oxMIF. The antibodies did not show any bystander effect such as complement-dependent cellular toxicity (CDC) or antibody-dependent cellular cytotoxicity (ADCC) (Hussain F. et al., Mol Cancer Ther. 2013 July; 12(7):1223-34).

Del Bano J. et al. provide a general review on bispecific antibodies for use in cancer immunotherapy (ANTIBODIES, vol. 5, no. 1, 2015, page 1).

In WO 2009/086920 A1 anti-MIF antibodies are described

WO 2016/156489 A1 refers to a dosage regimen of anti-MIF antibodies.

WO 2016/184886 A1 describes anti-MIF antibodies in the treatment of tumors containing mutant TP53 and mutant RAS.

KERSCHBAUMER R. J. et al. report neutralization of Macrophage Migration Inhibitory Factor (MIF) by fully human antibodies (JOURNAL OF BIOLOGICAL CHEMISTRY, vol. 287, no. 10, 2012, pages 7446-7455).

Douillard P. et al. disclose human antibodies specific for oxidized macrophage migration inhibitory factor (oxMIF) which synergize with chemotherapeutic agents in animal models of cancer” (Cancer Research, 2014, pages 2654-2654).

An urgent need exists for solving the problem on how to develop an immune cell mediated therapy, which has enhanced specificity and effectiveness. Specifically, there is an unmet need for overcoming limitations of therapeutic antibodies such as anti-oxMIF antibodies in oncology.

### SUMMARY OF THE INVENTION

It is the objective of the invention to provide for a bispecific antibody format directed against oxMIF and CD3 with improved biological activity.

The object is solved by the subject matter as claimed.

According to the invention there is provided an anti-oxMIF/anti-CD3 antibody comprising at least one binding site specifically recognizing oxMIF and at least one binding site specifically recognizing CD3.

The anti-oxMIF/anti-CD3 antibody of the invention has advantageous properties compared to the single antibody binding to oxMIF. Specifically, the bispecific formation of the inventive antibody brings tumor cells and T-cells in proximity to enable the T-cell to kill the tumor cells, thereby having the potential to significantly reduce tumor and metastasis burden.

According to a specific embodiment, the antibody induces T-cell-mediated cytotoxicity to a higher degree than the combination of anti-oxMIF and anti-CD3 antibodies. Such increase can be determined by any assay known in the art such as, but not limited to by a T cell Mediated Tumor Cell Lysis Assay. T-cell mediated cytotoxicity of the anti-oxMIF/anti-CD3 bispecific antibody may also be determined in vitro on cancer cells, specifically on solid tumor cells, specifically on colorectal, pancreatic, ovarian, lung cancer cells.

According to the invention, the oxMIF binding site is specific for oxidized MIF and does not bind to reduced MIF.

According to a specific embodiment, there is provided an anti-oxMIF/anti-CD3 antibody, wherein the binding site specifically recognizing oxMIF comprises

(a) a heavy chain variable region comprising

a CDR1-H1 sequence which has at least 70%, specifically at least 80%, at least 90%, at least 95%, more specifically at least 99% sequence identity to any of the sequences selected from the group consisting of SEQ ID NO 1, SEQ ID NO 7, SEQ ID NO 13, SEQ ID NO 19 and SEQ ID NO 26, and

a CDR2-H1 sequence which has at least 70%, specifically at least 80%, at least 90%, at least 95%, more specifically at least 99% sequence identity to any of the sequences selected from the group consisting of SEQ ID NO 2, SEQ ID NO 8, SEQ ID NO 14, SEQ ID NO 20 and SEQ ID NO 27, and

a CDR3-H1 sequence which has at least 70%, specifically at least 80%, at least 90%, at least 95%, more specifically at least 99% sequence identity to any of the sequences selected from the group consisting of SEQ ID NO 3, SEQ ID NO 9, SEQ ID NO 15 and SEQ ID NO 21, and

(b) a light chain variable region comprising

a CDR1-L1 sequence which has at least 70%, specifically at least 80%, at least 90%, at least 95%, more specifically at least 99% sequence identity to any of the sequences selected from the group consisting of SEQ ID N04, SEQ ID NO 10, SEQ ID NO 16, SEQ ID NO 22 and SEQ ID NO 28, and

a CDR2-L1 sequence which has at least 70%, specifically at least 80%, at least 90%, at least 95%, more specifically at least 99% sequence identity to any of the sequences selected from the group consisting of SEQ ID NO 5, SEQ ID NO 11, SEQ ID NO 17, SEQ ID NO 23 and SEQ ID NO 25, and

a CDR3-L1 sequence which has at least 70%, specifically at least 80%, at least 90%, at least 95%, more specifically at least 99% sequence identity to any of the sequences selected from the group consisting of SEQ ID NO 6, SEQ ID NO 12, SEQ ID NO 18 and SEQ ID NO 24.

According to an alternative embodiment there is provided an anti-oxMIF/anti-CD3 antibody as described herein comprising 0, 1 or 2 point mutations in each of the CDR sequences which are the

CDR1-H1 sequence selected from the group consisting of SEQ ID NO 1, SEQ ID NO 7, SEQ ID NO 13, SEQ ID NO 19 and SEQ ID NO 26, and

CDR2-H1 sequence selected from the group consisting of SEQ ID NO 2, SEQ ID NO 8, SEQ ID NO 14, SEQ ID NO 20 and SEQ ID NO 27, and

CDR3-H1 sequence selected from the group consisting of SEQ ID NO 3, SEQ ID NO 9, SEQ ID NO 15 and SEQ ID NO 21, and

CDR1-L1 sequence selected from the group consisting of SEQ ID N04, SEQ ID NO 10, SEQ ID NO 16, SEQ ID NO 22 and SEQ ID NO 28, and

CDR2-L1 sequence selected from the group consisting of SEQ ID NO 5, SEQ ID NO 11, SEQ ID NO 17, SEQ ID NO 23 and SEQ ID NO 25, and

CDR3-L1 sequence selected from the group consisting of SEQ ID NO 6, SEQ ID NO 12, SEQ ID NO 18 and SEQ ID NO 24.

According to a further embodiment, there is provided an anti-oxMIF/anti-CD3 antibody as described herein, wherein the binding site specifically recognizing CD3 comprises

(a) a heavy chain variable region comprising

a CDR1-H2 sequence which has at least 70% sequence identity to any of the sequences selected from the group consisting of SEQ ID NO 77, SEQ ID NO 86 and SEQ ID NO 92, and

a CDR2-H2 which has at least 70% sequence identity to any of the sequences selected from the group consisting of SEQ ID NO 78, SEQ ID NO 87, and SEQ ID NO 93, and

a CDR3-H2 which has at least 70% sequence identity to any of the sequences selected from the group consisting of SEQ ID NO 79, SEQ ID NO 88, SEQ ID NO 94, and SEQ ID NO 149, and

(b) a light chain comprising

a CDR1-L2 which has at least 70% sequence identity to any of the sequences selected from the group consisting of SEQ ID NO 80, SEQ ID NO 83, SEQ ID NO 89 and SEQ ID NO 95, and

a CDR2-L2 which has at least 70% sequence identity to any of the sequences selected from the group consisting of SEQ ID NO 81, SEQ ID NO 84, SEQ ID NO 90 and SEQ ID NO 96, and

a CDR3-L2 which has at least 70% sequence identity to any of the sequences selected from the group consisting of SEQ ID NO 82, SEQ ID NO 85, SEQ ID NO 91, SEQ ID NO 9, and SEQ ID NO 151.

According to a further embodiment, there is provided an anti-oxMIF/anti-CD3 antibody as described herein, said antibody comprising 0, 1, or 2 point mutations in each of the CDR sequences which are the

CDR1-H2 sequence from the group consisting of SEQ ID NO 77, SEQ ID NO 86 and SEQ ID NO 92, and

CDR2-H2 sequence from the group consisting of SEQ ID NO 78, SEQ ID NO 87, and SEQ ID NO 93, and

CDR3-H2 sequence from the group consisting of SEQ ID NO 79, SEQ ID NO 88, SEQ ID NO 94, and SEQ ID NO 149, and

CDR1-L2 sequence from the group consisting of SEQ ID NO 80, SEQ ID NO 83, SEQ ID NO 89 and SEQ ID NO 95, and

CDR2-L2 sequence from the group consisting of SEQ ID NO 81, SEQ ID NO 84, SEQ ID NO 90 and SEQ ID NO 96, and

CDR3-L2 sequence from the group consisting of SEQ ID NO 82, SEQ ID NO 85, SEQ ID NO 91, SEQ ID NO 97, and SEQ ID NO 151.

According to a specific embodiment, the invention specifically contemplates the use of any antibody comprising an oxMIF binding site derived from the sequences CDR1-H, CDR2-H, CDR3-H of the heavy chain variable region and/or the sequences CDR1-L, CDR2-L, CDR3-L of the light chain variable region, including constructs comprising single variable domains comprising either the combination of the CDR1-H, CDR2-H, CDR3-H sequences, or the combination of the CDR1-L, CDR2-L, CDR3-L sequences, or pairs of such variable domains, e.g. VH, VHH or VH/VL domain pairs.

According to a specific embodiment, the invention specifically contemplates the use of any antibody comprising a CD3 binding site derived from the sequences CDR1-H, CDR2-H, CDR3-H of the heavy chain variable region and/or the sequences CDR1-L, CDR2-L, CDR3-L of the light chain variable region, including constructs comprising single variable domains comprising either the combination of the CDR1-H, CDR2-H, CDR3-H sequences, or the combination of the CDR1-L, CDR2-L, CDR3-L sequences, or pairs of such variable domains, e.g. VH, VHH or VH/VL domain pairs.

Specific embodiments refer to the antibody comprising at least one of the CDR sequences of anti-oxMIF, preferably at least two or three, and at least one of the CDR sequences of anti-CD3.

Further specific embodiments refer to the antibody comprising at least one of the CDR sequences of anti-CD3, preferably at least two or three, and at least one of the CDR sequences of anti-oxMIF.

A further specific embodiment refers to the anti-oxMIF/anti-CD3 antibody wherein the corresponding variable heavy chain regions (VH) and the corresponding variable light chain regions (VL) regions are arranged, from N-terminus to C-terminus, in the order, VH(oxM IF)-VL(oxM IF)-VH(CD3)-VL(CD3), VH(CD3)-VL(CD3)-VH(oxMIF)-VL(oxMIF) or VH(CD3)-VL(CD3)-VL(oxMIF)-VH(oxMIF).

According to a specific embodiment, there is provided an anti-oxMIF/anti-CD3 antibody, comprising the sequences SEQ ID NO 7, SEQ ID NO 8, SEQ ID NO 9, SEQ ID NO 10, SEQ ID NO 11, SEQ ID NO 12, SEQ ID NO 77, SEQ ID NO 78, SEQ ID NO 149, SEQ ID NO 83, SEQ ID NO 84, and SEQ ID NO 151.

In a further embodiment, there is provided an anti-oxMIF/anti-CD3 antibody as described herein, wherein the binding site specifically recognizing oxMIF comprises a heavy chain variable region comprising the amino acid sequence of SEQ ID NO 172, or a sequence having at least 70% preferably at least 80%, preferably at least 90%, more preferably at least 95% sequence identity to SEQ ID NO 172, and a light chain variable region comprising the amino acid sequence of SEQ ID NO 134, or a sequence having at least 70% sequence identity to SEQ ID NO 134.

In an alternative embodiment, there is provided an anti-oxMIF/anti-CD3 antibody as described herein, wherein the binding site specifically recognizing oxMIF comprises a heavy chain variable region comprising the amino acid sequence of SEQ ID NO 172, or SEQ ID NO 172 comprising 0, 1, 2, 3, 4 or 5 point mutations, and a light chain variable region comprising the amino acid sequence of SEQ ID NO 134, or SEQ ID NO 134 comprising 0, 1, 2, 3, 4 or 5 point mutations.

Further provided herein is an anti-oxMIF/anti-CD3 antibody, wherein the binding site specifically recognizing CD3 comprises a heavy chain variable region having at least 70%, preferably at least 80%, preferably at least 90%, more preferably at least 95% sequence identity to the amino acid sequence of SEQ ID NO 135 and a light chain variable region having at least 70%, preferably at least 80%, preferably at least 90%, more preferably at least 95% sequence identity to the amino acid sequence of SEQ ID NO 136.

In an alternative embodiment, there is provided an anti-oxMIF/anti-CD3 antibody as described herein, wherein the binding site specifically recognizing CD3 comprises a heavy chain variable region comprising the amino acid sequence of SEQ ID NO 135, or SEQ ID NO 135 comprising 0, 1, 2, 3, 4 or 5 point mutations, and a light chain variable region comprising the amino acid sequence of SEQ ID NO 136, or SEQ ID NO 136 comprising 0, 1, 2, 3, 4 or 5 point mutations.

According to a further embodiment, there is provided an anti-oxMIF/anti-CD3 antibody described herein, wherein the at least one binding site is an antibody fragment selected from the group consisting of scFv, (scFv)2, scFvFc, Fab, Fab′, and F(ab′)2, fusion proteins of two single chain antibodies of different species (BiTE), minibody, TandAb, DutaMab, DART, and CrossMab.

According to a further embodiment, the antibody comprises at least one antibody domain which is of human origin, or a chimeric, or humanized antibody domain of mammalian origin other than human, preferably of humanized, murine or camelid origin. In a specific embodiment, the antibody is a nanobody, such as a single-domain antigen-binding fragment derived from camelid heavy-chain antibodies.

According to a further embodiment, the antibody as described herein comprises a monovalent, bivalent, trivalent, tetravalent, or multivalent binding site specifically binding oxMIF and a monovalent, bivalent, trivalent, tetravalent or multivalent binding site specifically binding CD3.

In a further embodiment, the antibody is a bispecific antibody, specifically selected from the group consisting of bispecific IgG, IgG appended with a CD3 binding site, BsAb fragments, bispecific fusion proteins, BsAb conjugates.

According to a further embodiment, herein provided is also a pharmaceutical composition comprising the anti-oxMIF/anti-CD3 antibody and a pharmaceutically acceptable carrier or excipient.

Specifically, the antibody or the pharmaceutical composition as described herein is provided for use in the treatment of a hyperproliferative disorder, specifically cancer involving any tissue or organ, specifically in the treatment of head, neck, breast, liver, skin, gastric, bladder, renal, esophageal, gynecological, bronchial, nasopharynx, thyroid, prostate, colorectal, ovarian, pancreas, lung cancers, and fibrosarcoma.

Specifically, the antibody as described herein can be used as a medicament.

Specifically, a method for the treatment of a hypoproliferative disease, specifically cancer is provided, comprising administering a therapeutically effective amount of a pharmaceutical composition as described herein to a subject in need thereof.

Further provided herein are isolated nucleic acid molecules encoding an anti-oxMIF/anti-CD3 antibody format of the invention.

In a further embodiment, there is provided an expression vector comprising nucleic acid molecule(s) as described herein.

A further embodiment refers to a host cell comprising said vector.

Further provided herein is a method of producing the anti-oxMIF/anti-CD3 antibody of the invention, comprising expressing a nucleic acid encoding the antibody in a host cell.

According to a specific embodiment, there is provided an in vitro method of detecting cellular expression of oxMIF, the method comprising: contacting a biological sample comprising a human cell to be tested with an anti-oxMIF/anti-CD3 antibody of the invention; and detecting binding of said antibody; wherein the binding of said antibody indicates the presence of oxMIF on a cell surface, to thereby detect whether the cell expresses oxMIF.

Specifically, the biological sample comprises intact human cells, biopsies, resections, tissue samples, or a membrane fraction of the cells to be tested.

More specifically, the anti-oxMIF/anti-CD3 antibody is labeled with a detectable label selected from the group consisting of a radioisotope, a fluorescent label, a chemiluminescent label, an enzyme label, and a bioluminescent label.

According to another aspect, the antibody conjugated to a detectable label can be used in diagnosing a hypoproliferative disease such as cancer, wherein the cells of a subject are expressing oxMIF.

### FIGURES

FIG. 1: Schematic picture of the anti-oxMIF/anti-CD3 bispecific antibody of oxMIF and CD3 that brings T cell in close proximity to tumor cell.

FIG. 2: Detection of oxMIF (vs. redMIF) with anti-oxMIF/CD3 bispecific antibodies by ELISA (C0008=anti-oxMIF monospecific control antibody).

FIG. 3: Binding of anti-oxMIF/CD3 bispecific antibodies to oxMIF and CD3. (C0008=anti-oxMIF monospecific control antibody).

FIG. 4: Detection of native CD3 on CD3-positive Jurkat t cells with anti-oxMIF/CD3 bispecific antibodies (C0008=anti-oxMIF monospecific control antibody).

FIG. 5 Binding of anti-oxMIF/CD3 bispecific entities to immobilized MIF (oxMIF) in an ELISA.

FIG. 6: Binding of anti-oxMIF/CD3 bispecific antibodies to native oxMIF on the cell surface of A2780 ovarian cancer cells (C0008=anti-oxMIF monospecific control antibody).

FIG. 7: Activation of t cells by anti-oxMIF/CD3 BiTE in the presence or absence of A2780 ovarian cancer cells (C0008=anti-oxMIF monospecific control antibody).

FIG. 8: PBMC mediated tumor cell killing of A2780 ovarian cancer cells (A) and A549 lung cancer cells (B) with anti-oxMIF/CD3 bispecific antibody C0006.

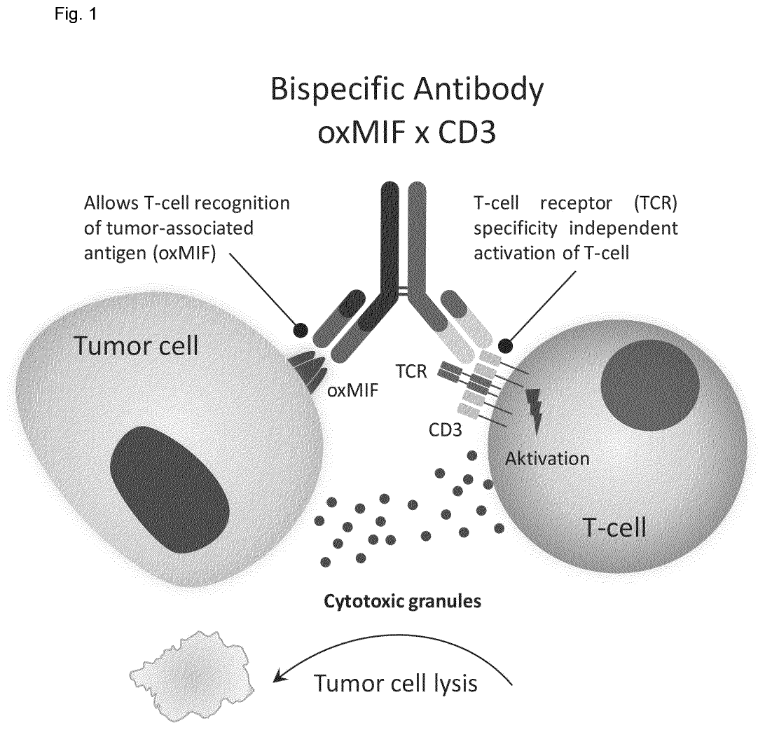

### DETAILED DESCRIPTION OF THE INVENTION

The term “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

The antibody of the invention comprises at least one binding site specifically recognizing oxMIF and at least one binding site specifically recognizing CD3.

The oxMIF binding site is specific for the oxidized form of MIF, i.e. specifically for human oxMIF but does not show substantial cross-reactivity to reduced MIF. oxMIF is the disease-related structural isoform of MIF which can be specifically and predominantly detected in the circulation of subjects with inflammatory diseases and in tumor tissue of cancer patients. In one embodiment, the humanized or human anti-oxMIF binding site comprises one or more (e.g., all three) light chain complementary determining regions of a humanized or human anti-oxMIF binding domain described herein, and/or one or more (e.g., all three) heavy chain complementary determining regions of a humanized or human anti-oxMIF binding domain described herein, e.g., a humanized or human anti-oxMIF binding domain comprising one or more, e.g., all three, LC CDRs and one or more, e.g., all three, HC CDRs.

The antibody of the invention further comprises at least one binding site specifically recognizing an epitope of CD3, specifically an epitope of human CD3, including the CD3γ (gamma) chain, CD3δ (delta) chain, and two CD3ϵ (epsilon) chains which are present on the cell surface. Clustering of CD3 on T cells, such as by immobilized anti-CD3 antibodies leads to T cell activation similar to the engagement of the T cell receptor but independent of its clone-typical specificity. In certain embodiments, the CD3 binding domain of the antibody described herein exhibits not only potent CD3 binding affinities with human CD3, but shows also excellent crossreactivity with the respective cynomolgus monkey CD3 proteins. In some instances, the CD3 binding domain of the antibody is cross-reactive with CD3 from cynomolgus monkey. In one embodiment, the anti-CD3 binding site comprises one or more (e.g., all three) light chain complementary determining regions of an anti-CD3 binding domain described herein, and/or one or more (e.g., all three) heavy chain complementary determining regions of an anti-CD3 binding domain described herein, e.g., an anti-CD3 binding domain comprising one or more, e.g., all three, LC CDRs and one or more, e.g., all three, HC CDRs.

The term “antibody” herein is used in the broadest sense and encompasses polypeptides or proteins that consist of or comprise antibody domains, which are understood as constant and/or variable domains of the heavy and/or light chains of immunoglobulins, with or without a linker sequence. The term encompasses various antibody structures, including but not limited to monoclonal antibodies, polyclonal antibodies, multispecific antibodies such as bispecific antibodies, and antibody fragments as long as they exhibit the desired antigen-binding activity, i.e. binding to oxMIF and CD3 epitopes.

Antibody domains may be of native structure or modified by mutagenesis or derivatization, e.g. to modify the antigen binding properties or any other property, such as stability or functional properties, such as binding to the Fc receptors, such as FcRn and/or Fc-gamma receptor. Polypeptide sequences are considered to be antibody domains, if comprising a beta-barrel structure consisting of at least two beta-strands of an antibody domain structure connected by a loop sequence.

It is understood that the term “antibody” includes derivatives thereof. A derivative is any combination of one or more antibody domains or antibodies of the invention and or a fusion protein in which any domain of the antibody of the invention may be fused at any position of one or more other proteins, such as other antibodies or antibody formats, e.g. a binding structure comprising CDR loops, a receptor polypeptide, but also ligands, scaffold proteins, enzymes, labels, toxins and the like.

The term “antibody” shall particularly refer to polypeptides or proteins that exhibit bispecific binding properties, i.e. to the target antigens oxMIF and CD3.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, Fab-scFv fusion, Fab-(scFv)₂-fusion, Fab-scFv-Fc, F(ab′)₂, ScFvFc, diabodies, cross-Fab fragments; linear antibodies; single-chain antibody molecules (e.g. scFv); and multispecific antibodies formed from antibody fragments. In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies. Antibody fragments as referred herein also encompass Fc domains comprising one or more structural loop regions containing antigen binding regions such as Fcab™ or full length antibody formats with IgG structures in which the Fc region has been replaced by an Fcab containing second distinct antigen binding site.

As used herein, “Fab fragment” refers to an antibody fragment comprising a light chain fragment comprising a VL domain and a constant domain of a light chain (CL), and a VH domain and a first constant domain (CH1) of a heavy chain. The bispecific antibodies of the invention can comprise at least one Fab fragment, wherein either the variable regions or the constant regions of the heavy and light chain are exchanged. Due to the exchange of either the variable regions or the constant regions, said Fab fragment is also referred to as “cross-Fab fragment” or “crossover Fab fragment”. Two different chain compositions of a crossover Fab molecule are possible and comprised in the antibodies of the invention: The variable regions of the Fab heavy and light chain can be exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab (VLVH). When the constant regions of the Fab heavy and light chain are exchanged, the crossover Fab molecule can comprise a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab (CLCH1).

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker can have the following orders in N-terminal to C-terminal direction: VH-CH1-linker-VL-CL, VL-CL-linker-VH-CH1, VH-CL-linker-VL-CH1 or VL-CH1-linker-VH-CL; and wherein said linker is a polypeptide of at least 20 amino acids, at least 30 amino acids, specifically between 32 and 50 amino acids. Said single chain Fab fragments VH-CH1-linker-VL-CL, VL-CL-linker-VH-CH1, VH-CL-linker-VL-CH1 and VL-CH1-linker-VH-CL, can be stabilized via the natural disulfide bond between the CL domain and the CH1 domain. In addition, these single chain Fab molecules might be further stabilized by generation of interchain disulfide bonds via insertion of cysteine residues.

The term “N-terminus” denotes the last amino acid of the N-terminus.

The term “C-terminus” denotes the last amino acid of the C-terminus.

A “BiTE” of “bi-specific T-cell engager” refers to an artificial monoclonal antibody which is a fusion protein consisting of two single-chain variable fragments (scFvs) of different antibodies, or amino acid sequences from four different genes, on a single peptide chain of about 50 kilodaltons. One of the scFvs binds to a T cell via the CD3 receptor, and the other to a tumor cell via oxMIF. Specifically, the BiTE is of about 50 kDa.

In a specific embodiment, the anti-oxMIF/anti-CD3 BiTE comprises the sequence or a sequence with at least 70%, specifically 75%, 80%, 85%, 90%, 95/ or 99% sequence identity:

(SEQ ID NO 137)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKGGGGSGGGGSGGGGSEVQLLESGGGLVQPGGSLRLSCAASGFT
FSIYSMNWVRQAPGKGLEWVSSIGSSGGTTYYADSVKGRFTISRDNSKNT
LYLQMNSLRAEDTAVYYCAGSQWLYGMDVWGQGTTVTVSSGGGGSDIKLQ
QSGAELARPGASVKMSCKTSGYTFTRYTMHWVKQRPGQGLEWIGYINPSR
GYTNYNQKFKDKATLTTDKSSSTAYMQLSSLTSEDSAVYYCARYYDDHYC
LDYWGQGTTLTVSSVEGGSGGSGGSGGSGGVDDIQLTQSPAIMSASPGEK
VTMTCRASSSVSYMNWYQQKSGTSPKRWIYDTSKVASGVPYRFSGSGSGT
SYSLTISSMEAEDAATYYCQQWSSNPLTFGAGTKLELKHHHHHH.

The term “minibody” refers to an antibody which is composed of a pair of single-chain Fv fragments which are linked via CH3 domains (single chain Fv-CH3), and Fvs with distinct specificity, which paired to the former part through heterodimerization process (Hu S. Z. et al., 1996, Cancer Research, 56, 3055-3061). To promote the heterodimerization efficiency, single-residue mutations can be introduced into each CH3 domains to achieve a “knobs and holes” approach. Far more than that, additional cysteine residues can also be introduced into CH3 domains to stabilize the bispecific minibody structure. A version of a minibody, Tribi minibody comprises a chain, which is designed to recognize antigens via its two Fv fragments, while the other chain possessing a Fv fragment takes charge of recruiting effector cells, such as T cytotoxic cells or NK cells. With the addition of this extra binding domain, the avidity of Tribi minibody is higher than that of the bispecific minibody. Specifically, the minibody is of about 75 kDa.

The term “nanobody” refers to a single-domain antibody (sdAb) fragment, i.e. an antibody fragment consisting of a single monomeric variable antibody domain. Nanobodies have a molecular weight of about 12-15 kDa.

The term “DART” refers to dual-affinity re-targeting antibodies which are the simplest form of bispecific antibodies (BsAb). A DART molecule consists of two engineered Fv fragments which have their own VH exchanged with the other one. The Fv1 is consisted of a VH from antibody A and a VL from antibody B, while the Fv2 is consisted of VH from Ab-B and VL from Ab-A. This inter-exchange of Fv domains releases variant fragments from the conformational constraint by the short linking peptide.

The term “DutaMab” refers to DutaMab, a format of BsAbs, which is formed by linking two independent paratopes in a single immunoglubin chain.

The term “TandAb” or “tandem antibody” refers to antibodies having in tandem two VLs/VHs pairs from two distinct Fv, which also means tandem antibodies do not carry Fc domains. TandAbs are smaller than whole IgGs or IgG-derived bispecific Abs but larger than single domain bispecific Abs. The moderate size also endows TandAb an increased tissue penetrating ability and longer serum half-life. In addition, the tetravalent property, which means bivalent for each antigen, can improve its binding efficiency and consequent therapeutic outcome. Specifically, the TandAb is of about 100 kDa.

In a specific embodiment, the anti-oxMIF/anti-CD3 TandAb of the invention comprises the sequence or a sequence with at least 70%, specifically 75%, 80%, 85%, 90%, 95/ or 99% sequence identity:

(SEQ ID NO 138)
DIQLTQSPAIMSASPGEKVTMTCRASSSVSYMNWYQQKSGTSPKRWIYDT
SKVASGVPYRFSGSGSGTSYSLTISSMEAEDAATYYCQQWSSNPLTFGAG
TKLELKGGSGGSEVQLLESGGGLVQPGGSLRLSCAASGFTFSIYSMNWVR
QAPGKGLEWVSSIGSSGGTTYYADSVKGRFTISRDNSKNTLYLQMNSLRA
EDTAVYYCAGSQWLYGMDVWGQGTTVTVSSGGSGGSDIQMTQSPSSLSAS
VGDRVTITCRSSQRIMTYLNWYQQKPGKAPKLLIFVASHSQSGVPSRFRG
SGSETDFTLTISGLQPEDSATYYCQQSFWTPLTFGGGTKVEIKGGSGGSD
IKLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYYD
DHYCLDYWGQGTTLTVSSHHHHHH.

The term “diabody” refers to a noncovalent dimer of single-chain Fv (scFv) fragment that consists of the heavy chain variable (V_{H}) and light chain variable (V_{L}) regions connected by a small peptide linker. Another form of diabody is single-chain (Fv)₂ in which two scFv fragments are covalently linked to each other. In addition, by tandem linking genes in each chain with internal linker, four VH and VL domains can be expressed in tandem and folded as single chain diabody (scDb), which is also an effective strategy for bispecific antibody production. Furthermore, fusing recombinant variable domains to an Fc region or CH3 domain (scDb-Fc and scDb-CH3, diabody-CH3) can double the valency of the final product. The increased size can also prolong the half-life of diabody in serum. Specifically, the diabody-CH3 is of about 125 kDa.

The term “crossMab” (where mab refers to monoclonal antibody) is a format of bispecific Abs derived from independent parental antibodies. Heavy chain mispairing is avoided by applying the knobs-into-holes (KIH) method. Light chain mispairing is avoided as the bispecific antibody is produced with antibody domain exchange whereas either the variable domains or the constant domains (CL and CH1) of one Fab arm are swapped between the light and heavy chains. This “crossover” keeps the antigen-binding affinity and also preserves the two different arms in order to avoid light-chain mispairing. Examples of CrossMabs can be, but are not limited to Fab, VH-VL and CH1-CL exchanged in different regions. In CrossMAbs Fabs the full VH-CH1 and VL-CL regions are exchanged; in CrossMAb VH-VL format only the VH and VL regions are exchanged; in CrossMAb CH1-CL1 format the CH1 and CL regions of bispecific antibody are exchanged. Specifically, the CrossMab is of about 150 kDa.

The term “IgG-scFv” refers to a kind of bispecific antibodies which is engineered for bispecificity by fusing two scFvs respectively to a monospecific IgG. The specificity of each scFv can be same or different. Furthermore, either the amino or the C terminus of each light or heavy chain can be appended with paired antibody variable domains, which leads to the production of diverse types of IgG-scFv BsAbs: IgG(H)-scFv or scFv-(H)IgG: IgG(H)-scFv, two scFvs with same specificity linked to the C terminus of the full-length IgG HC; scFv-(H)IgG, which is same like IgG(H)-scFv, except that the scFvs are linked to the HC N terminus. IgG(L)-scFv or scFv-(L)IgG: the two same scFvs connected to the C or N terminus of the IgG light chain, which forms the IgG(L)-scFv or scFv-(L)IgG, respectively. 2scFv-IgG or IgG-2scFv: generated by fusing two paired scFvs with different specificity to either the N terminus (2scFv-IgG) or the C terminus (IgG-2scFv). Specifically, the IgG(H)-scFv is about 200 kDa.

In a specific embodiment, the anti-oxMIF IgG×anti-CD3scFv fusion protein of the invention comprises the sequence or a sequence with at least 70%, specifically 75%, 80%, 85%, 90%, 95/ or 99% sequence identity with SEQ ID NO 139 and/or SEQ ID NO 140:

EVQLLESGGGLVQPGGSLRLSCAASGFTFSIYSMNWVRQAPGKGLEWVSS
IGSSGGTTYYADSVKGRFTISRDNSKNTLYLQMNSLRAEDTAVYYCAGSQ
WLYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGGGS
GGSGGSGGSGGSDIKLQQSGAELARPGASVKMSCKTSGYTFTRYTMHWVK
QRPGQGLEWIGYINPSRGYTNYNQKFKDKATLTTDKSSSTAYMQLSSLTS
EDSAVYYCARYYDDHYCLDYWGQGTTLTVSSVEGGSGGSGGSGGSGGVDD
IQLTQSPAIMSASPGEKVTMTCRASSSVSYMNWYQQKSGTSPKRWIYDTS
KVASGVPYRFSGSGSGTSYSLTISSMEAEDAATYYCQQWSSNPLTFGAGT
KLELK
(SEQ ID NO 139, anti-oxMIF heavy chain - anti-CD3
scFv fusion).
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(SEQ ID NO 140, Anti-oxMIF light chain).

In a specific embodiment, the anti-oxMIF/anti-CD3 Fab-scFv comprises the sequence or a sequence with at least 70%, specifically 75%, 80%, 85%, 90%, 95/ or 99% sequence identity:

(SEQ ID NO 173)
EVQLLESGGGLVQPGGSLRLSCAASGFTFSIYSMNWVRQAPGKGLEWVSS
IGSSGGTTYYADSVKGRFTISRDNSKNTLYLQMNSLRAEDTAVYYCAGSQ
WLYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCGGGGSGGGGSGGGGSQVQLVQSGAEVKKP
GASVKVSCKASGYTFTRYTMHWVRQAPGQGLEWMGYINPSRGYTNYNQKF
KDRVTLTTDKSSSTAYMELSSLRSEDTAVYYCARYYDDHYSLDYWGQGTL
VTVSSGGSGGSGGSGGSGGSDIQMTQSPSSLSASVGDRVTITCSASSSVS
YMNWYQQKPGKAPKRLIYDTSKLASGVPSRFSGSGSGTDFTLTISSLQPE
DFATYYCQQWSSNPFTFGQGTKLEIK.

In a specific embodiment, the anti-oxMIF/anti-CD3 Fab-scFv comprises the sequence or a sequence with at least 70%, specifically 75%, 80%, 85%, 90%, 95/ or 99% sequence identity to SEQ ID NO 154.

In a specific embodiment, the anti-oxMIF/anti-CD3 Fab—(scFv)2 comprises the sequence or a sequence with at least 70%, specifically 75%, 80%, 85%, 90%, 95/ or 99% sequence identity:

(SEQ ID NO 174)
EVQLLESGGGLVQPGGSLRLSCAASGFTFSIYSMNWVRQAPGKGLEWVSS
IGSSGGTTYYADSVKGRFTISRDNSKNTLYLQMNSLRAEDTAVYYCAGSQ
WLYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCGGGGSGGGGSGGGGSQVQLVQSGAEVKKP
GASVKVSCKASGYTFTRYTMHWVRQAPGQGLEWMGYINPSRGYTNYNQKF
KDRVTLTTDKSSSTAYMELSSLRSEDTAVYYCARYYDDHYSLDYWGQGTL
VTVSSGGSGGSGGSGGSGGSDIQMTQSPSSLSASVGDRVTITCSASSSVS
YMNWYQQKPGKAPKRLIYDTSKLASGVPSRFSGSGSGTDFTLTISSLQPE
DFATYYCQQWSSNPFTFGQGTKLEIK.

In a specific embodiment, the anti-oxMIF/anti-CD3 Fab—(scFv)2 comprises the sequence or a sequence with at least 70%, specifically 75%, 80%, 85%, 90%, 95/ or 99% sequence identity:

(SEQ ID NO 175)
DIQMTQSPSSLSASVGDRVTITCRSSQRIMTYLNWYQQKPGKAPKLLIFV
ASHSQSGVPSRFRGSGSETDFTLTISGLQPEDSATYYCQQSFWTPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGGGSGGGSGGGSQVQLVQSGAEVKKPGASVKVSCKA
SGYTFTRYTMHWVRQAPGQGLEWMGYINPSRGYTNYNQKFKDRVTLTTDK
SSSTAYMELSSLRSEDTAVYYCARYYDDHYSLDYWGQGTLVTVSSGGSGG
SGGSGGSGGSDIQMTQSPSSLSASVGDRVTITCSASSSVSYMNWYQQKPG
KAPKRLIYDTSKLASGVPSRFSGSGSGTDFTLTISSLQPEDFATYYCQQW
SSNPFTFGQGTKLEIK.

In a specific embodiment, the anti-oxMIF/anti-CD3 Fab-scFv-Fc comprises the sequence or a sequence with at least 70%, specifically 75%, 80%, 85%, 90%, 95/ or 99% sequence identity with SEQ ID NO 157, SEQ ID NO 158 and or SEQ ID NO 159.

In a specific embodiment, the anti-oxMIF/anti-CD3 IgG(Ic)-scFv comprises the sequence or a sequence with at least 70%, specifically 75%, 80%, 85%, 90%, 95/ or 99% sequence identity with SEQ ID NO 160 and/or SEQ ID NO 161.

In a specific embodiment, the anti-oxMIF/anti-CD3 Crossmab comprises the sequence or a sequence with at least 70%, specifically 75%, 80%, 85%, 90%, 95/ or 99% sequence identity with SEQ ID NO 162, SEQ ID NO 163, SEQ ID NO 164 and/or SEQ ID NO 165.

In a specific embodiment, the anti-oxMIF/anti-CD3 v IgG1-scFv comprises the sequence or a sequence with at least 70%, specifically 75%, 80%, 85%, 90%, 95/ or 99% sequence identity with SEQ ID NO 166 and/or SEQ ID NO 167

In a specific embodiment, the anti-oxMIF/anti-CD3 BiTE comprises the sequence or a sequence with at least 70%, specifically 75%, 80%, 85%, 90%, 95/ or 99% sequence identity with

(SEQ ID NO 176)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKGGGGSGGGGSGGGGSEVQLLESGGGLVQPGGSLRLSCAASGFTFSI
YSMNWVRQAPGKGLEWVSSIGSSGGTTYYADSVKGRFTISRDNSKNTLYLQ
MNSLRAEDTAVYYCAGSQWLYGMDVWGQGTTVTVSSGGGGSQVQLVQSGAE
VKKPGASVKVSCKASGYTFTRYTMHWVRQAPGQGLEWMGYINPSRGYTNYN
QKFKDRVTLTTDKSSSTAYMELSSLRSEDTAVYYCARYYDDHYSLDYWGQG
TLVTVSSGGSGGSGGSGGSGGSDIQMTQSPSSLSASVGDRVTITCSASSSV
SYMNWYQQKPGKAPKRLIYDTSKLASGVPSRFSGSGSGTDFTLTISSLQPE
DFATYYCQQWSSNPFTFGQGTKLEIK.

In a specific embodiment, the anti-oxMIF/anti-CD3 VL1-VH2-VL2-VH1, TandAb comprises the sequence or a sequence with at least 70%, specifically 75%, 80%, 85%, 90%, 95/ or 99% sequence identity with

(SEQ ID NO 177)
DIQMTQSPSSLSASVGDRVTITCSASSSVSYMNWYQQKPGKAPKRLIYDTS
KLASGVPSRFSGSGSGTDFTLTISSLQPEDFATYYCQQWSSNPFTFGQGTK
LEIKGGSGGSEVQLLESGGGLVQPGGSLRLSCAASGFTFSIYSMNWVRQAP
GKGLEWVSSIGSSGGTTYYADSVKGRFTISRDNSKNTLYLQMNSLRAEDTA
VYYCAGSQWLYGMDVWGQGTTVTVSSGGSGGSDIQMTQSPSSLSASVGDRV
TITCRSSQRIMTYLNWYQQKPGKAPKLLIFVASHSQSGVPSRFRGSGSETD
FTLTISGLQPEDSATYYCQQSFWTPLTFGGGTKVEIKGGSGGSQVQLVQSG
AEVKKPGASVKVSCKASGYTFTRYTMHWVRQAPGQGLEWMGYINPSRGYTN
YNQKFKDRVTLTTDKSSSTAYMELSSLRSEDTAVYYCARYYDDHYSLDYWG
QGTLVTVSS.

According to a specific embodiment, the antibodies described herein may comprise one or more tags for purification and/or detection, such as but not limited to affinity tags, solubility enhancement tags and monitoring tags.

Specifically, the affinity tag is selected from the group consisting of poly-histidine tag, poly-arginine tag, peptide substrate for antibodies, chitin binding domain, RNAse S peptide, protein A, β-galactosidase, FLAG tag, Strep II tag, streptavidin-binding peptide (SBP) tag, calmodulin-binding peptide (CBP), glutathione S-transferase (GST), maltose-binding protein (MBP), S-tag, HA tag, and c-Myc tag, specifically the tag is a His tag comprising one or more H, more specifically it is a hexahistidine tag.

By “fused” or “connected” is meant that the components (e.g. a Fab molecule and an Fc domain subunit) are linked by peptide bonds, either directly or via one or more peptide linkers.

The term “linker” as used herein refers to a peptide linker and is preferably a peptide with an amino acid sequence with a length of at least 5 amino acids, preferably with a length of 5 to 100, more preferably of 10 to 50 amino acids.

The term “immunoglobulin” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. An immunoglobulin of the IgG class essentially consists of two Fab molecules and an Fc domain, linked via the immunoglobulin hinge region. The heavy chain of an immunoglobulin may be assigned to one of five types, called α (IgA), δ (IgD), ϵ (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain.

The term “chimeric antibody” refers to an antibody in which a portion of the heavy and/or light chain is derived from a particular source or species, while the remainder of the heavy and/or light chain is derived from a different source or species, usually prepared by recombinant DNA techniques. Chimeric antibodies may comprise a rabbit or murine variable region and a human constant region. Other forms of “chimeric antibodies” are those in which the constant region has been modified or changed from that of the original antibody to generate the properties according to the invention. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art (Morrison, S. L., et al., Proc. Natl. Acad. Sci. 81 (1984) 6851-6855).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. As also mentioned for chimeric and humanized antibodies, the term “human antibody” as used herein also comprises such antibodies which are modified in the constant region e.g. by “class switching” i.e. change or mutation of Fc parts (e.g. from IgG1 to IgG4 and/or IgG1/IgG4 mutation.)

The term “recombinant human antibody”, as used herein, is intended to include all human antibodies that are prepared, expressed, created or isolated by recombinant means, such as antibodies isolated from a host cell such as a HEK cell, NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda, Md. (1991), vols. 1-3.

A “humanized” antibody refers to a chimeric antibody comprising amino acid residues from non-human HVRs and amino acid residues from human framework regions (FRs) which has undergone humanization. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. Other forms of humanized antibodies encompassed by the present invention are those in which the constant region has been additionally modified or changed from that of the original antibody to generate the new properties, e.g. in regard to C1q binding and/or Fc receptor (FcR) binding.

“Bispecific antibodies” according to the invention are antibodies which have two different binding specificities. Antibodies of the present invention are specific for oxMIF and CD3. The term bispecific antibody as used herein denotes an antibody or derivative or fragment thereof that has at least two binding sites each of which bind to different epitopes of oxMIF and CD3. Bispecific antibodies can be prepared as full length antibodies or antibody fragments as described herein. Examples of bispecific antibody formats can be, but are not limited to bispecific IgGs (BsIgG), IgGs appended with an additional antigen-binding moiety, BsAb fragments, bispecific fusion proteins, BsAb conjugates, hybrid bsIgGs, variable domain only bispecific antibody molecules, CH1/CL fusion proteins, Fab fusion proteins, modified Fc and CH3 fusion proteins, appended IgGs-HC fusions, appended IgGs-LC fusions, appended IgGs-HC& LC fusions, Fc fusions, CH3 fusions, IgE/IgM CH2 fusions, F(ab′)₂ fusions, CH1/CL, modified IgGs, non-immunoglobulin fusion proteins, Fc-modified IgGs, diabodies, etc. as described in Spiess C. et al., 2015, Mol. Immunol., 67, 95-106 and Brinkmann U. and Kontermann R. E., 2017, MABS, 9, 2, 182-212).

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site.

The term “epitope” as used herein shall in particular refer to a molecular structure which may completely make up a specific binding partner or be part of a specific binding partner to a binding site of an antibody format of the present invention. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding the polypeptide to form a tertiary structure and the amino acids are not necessarily adjacent to one another in the linear sequence. Such oxMIF epitope may be sequence EPCALCS (SEQ ID NO 145) located within the central region of oxMIF. However, the epitope may also be on the C-terminus of oxMIF.

The term “antigen binding domain” or “binding domain” or “binding-site” refers to the part of an antigen binding moiety that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “binding site” as used herein with respect to the antibody of the present invention refers to a molecular structure capable of binding interaction with an antigen. Typically, the binding site is located within the complementary determining region (CDR) of an antibody, herein also called “a CDR binding site”, which is a specific region with varying structures conferring binding function to various antigens. The varying structures can be derived from natural repertoires of antibodies, e.g. murine or human repertoires, or may be recombinantly or synthetically produced, e.g. by mutagenesis and specifically by randomization techniques. These include mutagenized CDR regions, loop regions of variable antibody domains, in particular CDR loops of antibodies, such as CDR1, CDR2 and CDR3 loops of any of VL and/or VH antibody domains. The antibody format as used according to the invention typically comprises one or more CDR binding sites, each specific to an antigen.

The term “specific” or “bispecific” as used herein shall refer to a binding reaction which is determinative of the cognate ligand of interest in a heterogeneous population of molecules. Herein, the binding reaction is at least with a CD3 antigen and an oxMIF antigen. Thus, under designated conditions, e.g. immunoassay conditions, the antibody that specifically binds to its particular target does not bind in a significant amount to other molecules present in a sample, specifically it does not show detectable binding to reduced MIF.

A specific binding site is typically not cross-reactive with other targets. Still, the specific binding site may specifically bind to one or more epitopes, isoforms or variants of the target, or be cross-reactive to other related target antigens, e.g., homologs or analogs.

The specific binding means that binding is selective in terms of target identity, high, medium or low binding affinity or avidity, as selected. Selective binding is usually achieved if the binding constant or binding dynamics to a target antigen such as oxMIF and CD3 is at least 10 fold different, preferably the difference is at least 100 fold, and more preferred a least 1000 fold compared to binding constant or binding dynamics to an antigen which is not the target antigen.

The bispecific antibody of the present invention specifically comprises two sites with specific binding properties, wherein two different target antigens, CD3 and oxMIF, are recognized by the antibody. Thus, an exemplary bispecific antibody format may comprise two binding sites, wherein each of the binding sites is capable of specifically binding a different antigen, CD3 and oxMIF.

The term “valent” as used within the current application denotes the presence of a specified number of binding sites in an antibody molecule. As such, the terms “bivalent”, “tetravalent”, and “hexavalent” denote the presence of two binding sites, four binding sites, and six binding sites, respectively, in an antibody molecule. The bispecific antibodies according to the invention are at least “bivalent” and may be “trivalent” or “multivalent” (e.g.“tetravalent” or “hexavalent”).

The term “monovalent” as used herein with respect to a binding site of an antibody shall refer to a molecule comprising only one binding site directed against a target antigen. The term “valency” is thus understood as the number of binding sites directed against the same target antigen, either specifically binding the same or different epitopes of an antigen.

The antibody of the present invention is understood to comprise a monovalent, bivalent, tetravalent or multivalent binding site specifically binding oxMIF and another monovalent, bivalent, tetravalent or multivalent binding site to specifically bind CD3.

According to a further embodiment, the antibody can comprise one or more additional binding sites specifically recognizing one or more antigens expressed on the effector T cells, specifically one or more of ADAM17, CD2, CD4, CD5, CD6, CD8, CD11a, CD11b, CD14, CD16, CD16b, CD25, CD28, CD30, CD32a, CD40, CD 40L, CD44, CD45, CD56, CD57, CD64, CD69, CD74, CD89, CD90, CD137, CD177, CEAECAM6, CEACAM8, HLA-Dra cahin, KIR, LSECtin or SLC44A2.

According to a specific embodiment, the antibody of the invention comprises one or more of the CD3 variable binding domains of otelixizumab, teplizumab, visilizumab or foralumab.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition (Kabat et al., 1991, Sequences of Proteins of Immunological Interest, 5th Ed. Public Health Service, National Institutes of Health, Bethesda, Md.) Hypervariable regions (HVRs) are also referred to as complementarity determining regions (CDRs), and these terms are used herein interchangeably in reference to portions of the variable region that form the antigen binding regions. The exact residue numbers which encompass a particular CDR will vary depending on the sequence and size of the CDR. Those skilled in the art can routinely determine which residues comprise a particular CDR given the variable region amino acid sequence of the antibody.

Kabat defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., 1983, U.S. Dept. of Health and Human Services, “Sequence of Proteins of Immunological Interest”. Unless otherwise specified, references to the numbering of specific amino acid residue positions in an antibody variable region are according to the Kabat numbering system. In a specific embodiment, the numbering of the constant region is according to EU numbering index.

CDRs also comprise “specificity determining residues,” or “SDRs,” which are residues that contact antigen. SDRs are contained within regions of the CDRs called abbreviated-CDRs, or a-CDRs. Unless otherwise indicated, HVR residues and other residues in the variable domain (e.g., FR residues) are numbered herein according to Kabat et al., supra.

According to a specific embodiment, the anti-CD3 binding site comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31 and any humanized derivatives thereof.

The antibody of the invention specifically comprises one or more of the sequences as described below:

TABLE 1
anti-oxMIF heavy chain sequences

| | HV- | | HV-CDR2 | | | |
|---|---|---|---|---|---|---|
| | CDR1 | | (CDR2- | | HV-CDR3 | |
| HV-FR1 | (CDR1-H1) | HV-FR2 | H1) | HV-FR3 | (CD3-H1) | HV-FR4 |
| EVQLLESGGG | IYTMD | WVRQA | YISPSGG | RFTISRDNSKNTL | RQYVLRY | WGQ |
| LVQPGGSLRL | SEQ ID | PGKGLE | NTSYADS | YLQMNSLRAEDT | FDWSAD | GTMV |
| SCAASGFTFS | NO 1 | WVS | VKG SEQ | AVYYCAS | AFDI | TVSS |
| SEQ ID NO 29 | | SEQ ID | ID NO 2 | SEQ ID NO 31 | SEQ ID NO | SEQ ID |
| | | NO 30 | | | 3 | NO 32 |
| EVQLLESGGG | IYSMN | WVRQA | SIGSSGG | RFTISRDNSKNTL | SQWLYG | WGQG |
| LVQPGGSLRL | SEQ ID | PGKGLE | TTYYADS | YLQMNSLRAEDT | MDV | TTVTV |
| SCAASGFTFS | NO 7 | WVS | VKG | AVYYCAG | SEQ ID NO | SS |
| SEQ ID NO 37 | | SEQ ID | SEQ ID | SEQ ID NO 39 | 9 | SEQ ID |
| | | NO 38 | NO 8 | | | NO 40 |
| EVQLLESGGG | KYYMI | WVRQA | WIGPSG | RFTISRDNSKNTL | GTPDYG | WGQG |
| LVQPGGSLRL | SEQ ID | PGKGL | GFTFYA | YLQMNSLRAEDT | GNSLDH | TLVTV |
| SCAASGFTFS | NO 13 | EWVS | DSVKG | AVYYCAR | SEQ ID NO | SS |
| SEQ ID NO 45 | | SEQ ID | SEQ ID | SEQ ID NO 47 | 15 | SEQ ID |
| | | NO 46 | NO 14 | | | NO 48 |
| EVQLLESGGG | IYAMD | WVRQA | GIVPSGG | RFTISRDNSKNTL | VNVIAVA | WGQ |
| LVQPGGSLRL | SEQ ID | PGKGL | FTKYADS | YLQMNSLRAEDT | GTGYYYY | GTTV |
| SCAASGFTFS | NO 19 | EWVS | VKG | AVYYCAR | GMDV | TVSS |
| SEQ ID NO 53 | | SEQ ID | SEQ ID | SEQ ID NO 55 | SEQ ID NO | SEQ ID |
| | | NO 54 | NO 20 | | 21 | NO 56 |
| EVQLLESGGG | IYAMD | WVRQA | GIVPSGG | RFTISRDNSKNTL | VNVIAVA | WGQ |
| LVQPGGSLRL | SEQ ID | PGKGL | FTKYADS | YLQMNSLRAEDT | GTGYYYY | GTTV |
| SCAASGFTFS | NO 19 | EWVS | VKG | AVYYCAR | GMDV | TVSS |
| SEQ ID NO 61 | | SEQ ID | SEQ ID | SEQ ID NO 63 | SEQ ID NO | SEQ ID |
| | | NO 62 | NO 20 | | 21 | NO 64 |
| EVQLLESGGG | WYAMD | WVRQA | GIYPSGG | RFTISRDNSKNTL | VNVIAVAG | WGQG |
| LVQPGGSLRL | SEQ ID | PGKGL | RTKYAD | YLQMNSLRAEDT | TGYYYYG | TTVTV |
| SCAASGFTFS | NO 26 | EWVS | SVKG | AVYYCAR | MDV | SS |
| SEQ ID NO 69 | | SEQ ID | SEQ ID | SEQ ID NO 71 | SEQ ID NO | SEQ ID |
| | | NO 70 | NO 27 | | 21 | NO 72 |

TABLE 2
anti-oxMIF light chain sequences

| | LV- | | LV- | | | |
|---|---|---|---|---|---|---|
| | CDR1 | | CDR2 | | LV-CDR3 | |
| | (CDR1- | | (CDR2- | | (CDR3- | LV- |
| LV-FR1 | L1) | LV-FR2 | L1) | LV-FR3 | L1) | FR4 |
| DIQMTQSPSSL | RASQSI | WYQQKP | AASSL | GVPSRFSGSGS | QQSYST | FGQG |
| SASVGDRVTIT | SSYLN | GKAPKLLI | QS | GTDFTLTISSLQ | PWT | TKVEI |
| C SEQ ID NO 33 | SEQ ID | Y SEQ ID | SEQ ID | PEDFATYYC | SEQ ID | K SEQ |
| | NO 4 | NO 34 | NO 5 | SEQ ID NO 35 | NO 6 | ID NO |
| | | | | | | 36 |
| DIQMTQSPSSL | RSSQRI | WYQQKP | VASHS | GVPSRFRGSGS | QQSFWT | FGGG |
| SASVGDRVTIT | MTYLN | GKAPKLLI | QS | ETDFTLTISGLQ | PLT | TKVEI |
| C SEQ ID NO 41 | SEQ ID | F SEQ ID | SEQ ID | PEDSATYYC | SEQ ID | K SEQ |
| | NO 10 | NO 42 | NO 11 | SEQ ID NO 43 | NO 12 | ID NO |
| | | | | | | 44 |
| DIQMTQSPSSL | RASQSI | WYQHKP | ATSRL | GVPSRFSGGGS | QQTYST | FGGG |
| PASVGDRVTIT | GTYLS | GNAPKLLI | QS | GTRFTLAISSLQ | PLT | TKVDI |
| C SEQ ID NO 49 | SEQ ID | Y SEQ ID | SEQ ID | PDDFATYFC | SEQ ID | K SEQ |
| | NO 16 | NO 50 | NO 17 | SEQ ID NO 51 | NO 18 | ID NO |
| | | | | | | 52 |
| DIQMTQSPGTL | RASQG | WYQQKP | GTSSR | GIPDRFSGSASG | QQYGRS | FGGG |
| SLSPGERATLS | VSSSSL | GQAPRLLI | AT | TDFTLTISRLQP | LT | TKVEI |
| C SEQ ID NO 57 | A SEQ | Y SEQ ID | SEQ ID | EDFAVYYC | SEQ ID | K SEQ |
| | ID NO 22 | NO 58 | NO 23 | SEQ ID NO 59 | NO 24 | ID NO |
| | | | | | | 60 |
| DIQMTQSPVTL | RASQS | WYQQKP | GASNR | GIPDRFSGSGS | QQYGNS | FGGG |
| SLSPGERATLS | VRSSYL | GQTPRLLI | AT | GTDFTLTISRLE | LT | TKVEI |
| C SEQ ID NO 65 | A SEQ | Y SEQ ID | SEQ ID | PEDFAVYYC | SEQ ID | K SEQ |
| | ID NO 22 | NO 66 | NO 25 | SEQ ID NO 67 | NO 24 | ID NO |
| | | | | | | 68 |
| DIQMTQSPGTL | RASQG | WYQQKP | GTSSR | GIPDRFSGSASG | QQYGRS | FGGG |
| SLSPGERATLS | VSSSSL | GQAPRLLI | AT | TDFTLTISRLQP | LT | TKVEI |
| C SEQ ID NO 73 | A SEQ | Y SEQ ID | SEQ ID | EDFAVYYC | SEQ ID | K SEQ |
| | ID NO 28 | NO 74 | NO 23 | SEQ ID NO 75 | NO 24 | ID NO |
| | | | | | | 76 |

TABLE 3
anti-CD3 heavy chain sequences

| | HV- | | | | HV- | |
|---|---|---|---|---|---|---|
| | CDR1 | | | | CDR3 | |
| | (CDR1- | | HV-CDR2 | | (CDR3- | |
| HV-FR1 | H2) | HV-FR2 | (CDR2-H2) | HV-FR3 | H2) | HV-FR4 |
| QVQLVQSGAE | RYTM | WVRQAP | YINPSRG | RVTLTTDKSSST | YYDDH | WGQGT |
| VKKPGASVKV | H | GQGLE | YTNYNQK | AYMELSSLRSED | YSLDY | LVTVSS |
| SCKASGYTFT | SEQ ID | WMG | FKD | TAVYYCAR | SEQ ID | SEQ ID |
| SEQ ID NO 146 | NO 77 | SEQ ID | SEQ ID NO | SEQ ID NO 148 | NO 149 | NO 101 |
| | | NO 147 | 78 | | | |
| DIKLQQSGAEL | RYTM | WVKQRP | YINPSRG | KATLTTDKSSST | YYDDH | WGQGT |
| ARPGASVKMS | H SEQ | GQGLE | YTNYNQK | AYMQLSSLTSED | YCLDY | TLTVSS |
| CKTSGYTFT | ID NO | WIG | FKD | SAVYYCAR | SEQ ID | SEQ ID |
| SEQ ID NO 98 | 77 | SEQ ID | SEQ ID NO | SEQ ID NO 100 | NO 79 | NO 101 |
| | | NO 99 | 78 | | | |
| QVQLQQSGAE | RYTM | WVKQRP | YINPSRG | KATLTTDKSSST | YYDDH | WGQGT |
| LARPGASVKM | H SEQ | GQGLE | YTNYNQK | AYMQLSSLTSED | YCLDY | TLTVSS |
| SCKASGYTFT | ID NO | WIG | FKD | SAVYYCAR | SEQ ID | SEQ ID |
| SEQ ID NO 106 | 77 | SEQ ID | SEQ ID NO | SEQ ID NO 100 | NO 79 | NO 101 |
| | | NO 99 | 78 | | | |
| QVQLVQSGGG | RYTM | WVRQAP | YINPSRG | RFTISRDNSKNT | YYDDH | WGQGT |
| VVQPGRSLRL | H SEQ | GKGLEW | YTNYNQK | AFLQMDSLRPED | YCLDY | PVTVSS |
| SCKASGYTFT | ID NO | IG | VKD | TGVYFCAR | SEQ ID | SEQ ID |
| SEQ ID NO 110 | 77 | SEQ ID | SEQ ID NO | SEQ ID NO 112 | NO 79 | NO 113 |
| | | NO 111 | 78 | | | |
| QVQLVESGGG | GYGM | WVRQAP | VIWYDGS | RFTISRDNSKNT | QMGY | WGRGT |
| VVQPGRSLRL | H SEQ | GKGLEW | KKYYVDS | LYLQMNSLRAED | WHFDL | LVTVSS |
| SCAASGFKFS | ID NO | VA | VKG | TAVYYCAR | SEQ ID | SEQ ID |
| SEQ ID NO 118 | 86 | SEQ ID | SEQ ID NO | SEQ ID NO 120 | NO 88 | NO 121 |
| | | NO 119 | 87 | | | |
| EVQLLESGGG | SFPMA | WVRQAP | TISTSGG | RFTISRDNSKNT | FRQYS | WGQGT |
| LVQPGGSLRL | SEQ ID | GKGLEW | RTYYRDS | LYLQMNSLRAED | GGFDY | LVTVSS |
| SCAASGFTFS | NO 92 | VS | VKG | TAVYYCAK | SEQ ID | SEQ ID |
| SEQ ID NO 126 | | SEQ ID | SEQ ID NO | SEQ ID NO 128 | NO 94 | NO 129 |
| | | NO 127 | 93 | | | |

TABLE 4
anti-CD3 light chain sequences

| | LV- | | | | | |
|---|---|---|---|---|---|---|
| | CDR1 | | | | | |
| | (CDR1- | | LV-CDR2 | | LV-CDR3 | LV- |
| LV-FR1 | L2) | LV-FR2 | (CDR2-L2) | LV-FR3 | (CDR3-L2) | FR4 |
| DIQMTQSPSSL | SASSSV | WYQQKP | DTSKLAS | GVPSRFSG | QQWSSN | FTFG |
| SASVGDRVTIT | SYMN | GKAPKRLI | SEQ ID NO | SGSGTDFTL | PSEQ ID | QGTK |
| C SEQ ID NO 33 | SEQ ID | Y SEQ ID | 84 | TISSLQPED | NO 151 | LEIK |
| | NO 83 | NO 150 | | FATYYC | | SEQ |
| | | | | SEQ ID NO | | ID NO |
| | | | | 35 | | 152 |
| DIQLTQSPAIM | RASSSV | WYQQKS | DTSKVAS | GVPYRFSG | QQWSSN | FGAG |
| SASPGEKVTM | SYMN | GTSPKRW | SEQ ID NO | SGSGTSYSL | PLT SEQ | TKLEL |
| TC SEQ ID NO | SEQ ID | IY SEQ | 81 | TISSMEAED | ID NO 82 | K SEQ |
| 102 | NO 80 | ID NO 103 | | AATYYC | | ID NO |
| | | | | SEQ ID NO | | 105 |
| | | | | 104 | | |
| QIVLTQSPAIM | SASSSV | WYQQKS | DTSKLAS | GVPAHFRG | QQWSSN | FGSG |
| SASPGEKVTM | SYMN | GTSPKRW | SEQ ID NO | SGSGTSYSL | PFT SEQ | TKLEI |
| TC SEQ ID NO | SEQ | IY SEQ | 84 | TISGMEAED | ID NO 85 | N SEQ |
| 107 | ID NO 83 | ID NO 103 | | AATYYC | | ID NO |
| | | | | SEQ ID NO | | 109 |
| | | | | 108 | | |
| DIQMTQSPSSL | SASSSV | WYQQTP | DTSKLAS | GVPSRFSG | QQWSSN | FGQG |
| SASVGDRVTIT | SYMN | GKAPKR | SEQ ID NO | SGSGTDYT | PFT SEQ | TKLQI |
| C SEQ ID NO | SEQ ID | WIY SEQ | 84 | FTISSLQPE | ID NO 85 | T SEQ |
| 114 | NO 83 | ID NO 115 | | DIATYYC | | ID NO |
| | | | | SEQ ID NO | | 117 |
| | | | | 116 | | |
| EIVLTQSPATL | RASQS | WYQQKP | DASNRAT | GIPARFSGS | QQRSNW | FGGG |
| SLSPGERATLS | VSSYLA | GQAPRLLI | SEQ ID NO | GSGTDFTLT | PPLT | TKVEI |
| C SEQ ID NO | SEQ ID | Y SEQ ID | 90 | ISSLEPEDF | SEQ ID NO | K SEQ |
| 122 | NO 89 | NO 123 | | AVYYC | 91 | ID NO |
| | | | | SEQ ID NO | | 125 |
| | | | | 124 | | |
| DIQLTQPNSVS | TLSSGN | WYQLYEG | DDDKRPD | GVPDRFSG | HSYVSSF | FGGG |
| TSLGSTVKLSC | IENNYV | RSPTTMIY | SEQ ID NO | SIDRSSNSA | NV | TKLTV |
| SEQ ID NO 130 | H SEQ | SEQ ID NO | 96 | FLTIHNVAIE | SEQ ID NO | L |
| | ID NO 95 | 131 | | DEAIYFC | 97 | SEQ |
| | | | | SEQ ID NO | | ID NO |
| | | | | 132 | | 133 |

The variable heavy chain sequence of the anti-oxMIF antibody can be as follows:

(SEQ ID NO 172)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSS.

The variable light chain sequence of the anti-oxMIF antibody can be as follows:

(SEQ ID NO 134)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIK.

The variable heavy chain sequence of the anti-CD3 antibody can be as follows:

(SEQ ID NO 135)
DIKLQQSGAELARPGASVKMSCKTSGYTFTRYTMHWVKQRPGQGLEWIGYI
NPSRGYTNYNQKFKDKATLTTDKSSSTAYMQLSSLTSEDSAVYYCARYYDD
HYCLDYWGQGTTLTVSS.

The variable light chain sequence of the anti-CD3 antibody can be as follows:

(SEQ ID NO 136)
DIQLTQSPAIMSASPGEKVTMTCRASSSVSYMNWYQQKSGTSPKRWIYDTS
KVASGVPYRFSGSGSGTSYSLTISSMEAEDAATYYCQQWSSNPLTFGAGTK
LELK.

Specifically, the ϵ chain of CD3 can comprise the sequence

(SEQ ID NO 141)
MQSGTHWRVLGLCLLSVGVWGQDGNEEMGGITQTPYKVSISGTTVILTCPQ
YPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSK
NRKAKAKPVTRGAGAGGRQRGQNKERPPPVPNPDYEPIRKGQRDLYSGLNQ
RRI.

Specifically, the δ chain of CD3 can comprise the sequence

(SEQ ID NO 142)
MEHSTFLSGLVLATLLSQVSPFKIPIEELEDRVFVNCNTSITWVEGTVGTL
LSDITRLDLGKRILDPRGIYRCNGTDIYKDKESTVQVHYRMCQSCVELDPA
TVAGIIVTDVIATLLLALGVFCFAGHETGRLSGAADTQALLRNDQVYQPLR
DRDDAQYSHLGGNWARNK.

Specifically, the γ chain of CD3 can comprise the sequence

(SEQ ID NO 143)
MEQGKGLAVLILAIILLQGTLAQSIKGNHLVKVYDYQEDGSVLLTCDAEAK
NITWFKDGKMIGFLTEDKKKWNLGSNAKDPRGMYQCKGSQNKSKPLQVYYR
MCQNCIELNAATISGFLFAEIVSIFVLAVGVYFIAGQDGVRQSRASDKQTL
LPNDQLYQPLKDREDDQYSHLQGNQLRRN.

According to a specific embodiment, the domain of oxMIF specifically recognized by the oxMIF binding site comprises the sequence

(SEQ ID NO 144)
MPMFIVNTNVPRASVPDGFLSELTQQLAQATGKPPQYIAVHVVPDQLMAFG
GSSEPCALCSLHSIGKIGGAQNRSYSKLLCGLLAERLRISPDRVYINYYDM
NAANVGWNNSTFA.

Specifically, any one of SEQ ID Nos 134 to SEQ ID NO 144 and SEQ ID NO 172 can comprise 1, 2, 3, or 4 point mutations.

A “point mutation” is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids. Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) sequence identity” with respect to the polypeptide sequences identified herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

According to the present invention, sequence identity of the CDR or framework region sequences is at least 70%, 75%, 80%, 85%, 90%, 95%, 96%, 97%, 98%, 99%, 99.5% or 100% with the respective sequences described herein.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-oxMIF/anti-CD3 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“No substantial cross-reactivity” means that a molecule (e.g., an antibody) does not recognize or specifically bind an antigen different from the actual target antigen of the molecule (e.g. an antigen closely related to the target antigen), specifically reduced MIF, particularly when compared to that target antigen. For example, an antibody may bind less than about 10% to less than about 5% to an antigen different from the actual target antigen, or may bind said antigen different from the actual target antigen at an amount consisting of less than about 10%, 9%, 8% 7%, 6%, 5%, 4%, 3%, 2%, 1%, 0.5%, 0.2%, or 0.1%, preferably less than about 2%, 1%, or 0.5%, and most preferably less than about 0.2% or 0.1% antigen different from the actual target antigen. Binding can be determined by any method known in the art such as, but not limited to ELISA or surface plasmon resonance.

The recombinant production of the antibody of the invention preferably employs an expression system, e.g. including expression constructs or vectors comprising a nucleotide sequence encoding the antibody format.

The term “expression system” refers to nucleic acid molecules containing a desired coding sequence and control sequences in operable linkage, so that hosts transformed or transfected with these sequences are capable of producing the encoded proteins. In order to effect transformation, the expression system may be included on a vector; however, the relevant DNA may then also be integrated into the host chromosome. Alternatively, an expression system can be used for in vitro transcription/translation.

“Expression vectors” used herein are defined as DNA sequences that are required for the transcription of cloned recombinant nucleotide sequences, i.e. of recombinant genes and the translation of their mRNA in a suitable host organism. Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences.

Specifically the term refers to a vehicle by which a DNA or RNA sequence (e.g. a foreign gene), e.g. a nucleotide sequence encoding the antibody format of the present invention, can be introduced into a host cell, so as to transform the host and promote expression (e.g. transcription and translation) of the introduced sequence. Plasmids are preferred vectors of the invention.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites.

A “cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA that can readily accept additional (foreign) DNA and which can readily be introduced into a suitable host cell. A vector of the invention often contains coding DNA and expression control sequences, e.g. promoter DNA, and has one or more restriction sites suitable for inserting foreign DNA. Coding DNA is a DNA sequence that encodes a particular amino acid sequence for a particular polypeptide or protein such as an antibody format of the invention. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors of the invention will often include one or more replication systems for cloning or expression, one or more markers for selection in the host, e.g. antibiotic resistance, and one or more expression cassettes.

The procedures used to ligate DNA sequences, e.g. providing or coding for the factors of the present invention and/or the protein of interest, a promoter, a terminator and further sequences, respectively, and to insert them into suitable vectors containing the information necessary for integration or host replication, are well known to persons skilled in the art, e.g. described by J. Sambrook et al., “Molecular Cloning 2nd ed.”, Cold Spring Harbor Laboratory Press (1989).

A host cell is specifically understood as a cell, a recombinant cell or cell line transfected with an expression construct, such as a vector according to the invention.

The term “host cell line” as used herein refers to an established clone of a particular cell type that has acquired the ability to proliferate over a prolonged period of time. The term host cell line refers to a cell line as used for expressing an endogenous or recombinant gene to produce polypeptides, such as the recombinant antibody format of the invention.

A “production host cell” or “production cell” is commonly understood to be a cell line or culture of cells ready-to-use for cultivation in a bioreactor to obtain the product of a production process, the recombinant antibody format of the invention. The host cell type according to the present invention may be any prokaryotic or eukaryotic cell.

The term “recombinant” as used herein shall mean “being prepared by genetic engineering” or “the result of genetic engineering”, e.g. specifically employing heterologous sequences incorporated in a recombinant vector or recombinant host cell.

A bispecific antibody of the invention may be produced using any known and well-established expression system and recombinant cell culturing technology, for example, by expression in bacterial hosts (prokaryotic systems), or eukaryotic systems such as yeasts, fungi, insect cells or mammalian cells. An antibody molecule of the present invention may be produced in transgenic organisms such as a goat, a plant or a transgenic mouse, an engineered mouse strain that has large fragments of the human immunoglobulin loci and is deficient in mouse antibody production. An antibody may also be produced by chemical synthesis.

According to a specific embodiment, the host cell is a production cell line of cells selected from the group consisting of CHO, PerC6, CAP, HEK, HeLa, NS0, SP2/0, hybridoma and Jurkat. More specifically, the host cell is obtained from HEK293 cells.

The host cell of the invention is specifically cultivated or maintained in a serum-free culture, e.g. comprising other components, such as plasma proteins, hormones, and growth factors, as an alternative to serum.

Host cells are most preferred, when being established, adapted, and completely cultivated under serum free conditions, and optionally in media which are free of any protein/peptide of animal origin.

Anti-oxMIF/anti-CD3 antibodies can be recovered from the culture medium using standard protein purification methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

As used herein, “treatment”, “treat” or “treating” refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The anti-oxMIF/anti-CD3 antibody of the invention and the pharmaceutical compositions comprising it, can be administered in combination with one or more other therapeutic, diagnostic or prophylactic agents. Additional therapeutic agents include other anti-neoplastic, antitumor, anti-angiogenic, chemotherapeutic agents, steroids, or checkpoint inhibitors depending on the disease to be treated.

The pharmaceutical compositions of this invention may be in a variety of forms, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. As will be appreciated by the skilled artisan, the route and/or mode of administration will vary depending upon the desired results.

The anti-oxMIF/anti-CD3 antibody may be administered once, but more preferably is administered multiple times. For example, the antibody may be administered from three times daily to once every six months or longer. The administering may be on a schedule such as three times daily, twice daily, once daily, once every two days, once every three days, once weekly, once every two weeks, once every month, once every two months, once every three months and once every six months.

The term “cancer” as used herein refers to proliferative diseases, specifically to solid cancers, such as colorectal cancer, ovarian cancer, pancreas cancer, lung cancer, melanoma, squamous cell carcinoma (SCC) (e.g., head and neck, esophageal, and oral cavity), hepatocellular carcinoma, colorectal adenocarcinoma, kidney cancer, medullary thyroid cancer, papillary thyroid cancer, astrocytic tumor, neuroblastoma, Ewing's sarcoma, cervical cancer, endometrial carcinoma, breast cancer, prostate cancer, and malignant seminoma, including refractory versions of any of the above cancers, or a combination of one or more of the above cancers.

Detection of cellular expression of oxMIF can be performed with the antibody as described herein, said antibody being labeled so that specific expression of oxMIF can be detected. Antibody labelling can be performed according to methods well known in the art. Such labels can be, but are not limited to radioisotopes, fluorescent labels, chemiluminescent labels, enzyme labels, and bioluminescent labels.

The invention further encompasses following items:

1. An anti-oxMIF/anti-CD3 antibody comprising at least one binding site specifically recognizing oxMIF and at least one binding site specifically recognizing CD3.

2. The anti-oxMIF/anti-CD3 antibody of item 1, wherein the binding site specifically recognizing oxMIF comprises

(a) a heavy chain variable region comprising

a CDR1-H1 sequence which has at least 70% sequence identity to any of the sequences selected from the group consisting of SEQ ID NO 1, SEQ ID NO 7, SEQ ID NO 13, SEQ ID NO 19 and SEQ ID NO 26, and

a CDR2-H1 sequence which has at least 70% sequence identity to any of the sequences selected from the group consisting of SEQ ID NO 2, SEQ ID NO 8, SEQ ID NO 14, SEQ ID NO 20 and SEQ ID NO 27, and

a CDR3-H1 sequence which has at least 70% sequence identity to any of the sequences selected from the group consisting of SEQ ID NO 3, SEQ ID NO 9, SEQ ID NO 15 and SEQ ID NO 21, and

(b) a light chain variable region comprising

a CDR1-L1 sequence which has at least 70% sequence identity to any of the sequences selected from the group consisting of SEQ ID N04, SEQ ID NO 10, SEQ ID NO 16, SEQ ID NO 22 and SEQ ID NO 28, and

a CDR2-L1 sequence which has at least 70% sequence identity to any of the sequences selected from the group consisting of SEQ ID NO 5, SEQ ID NO 11, SEQ ID NO 17, SEQ ID NO 23 and SEQ ID NO 25, and

a CDR3-L1 sequence which has at least 70% sequence identity to any of the sequences selected from the group consisting of SEQ ID NO 6, SEQ ID NO 12, SEQ ID NO 18 and SEQ ID NO 24.

3. The anti-oxMIF/anti-CD3 antibody of item 1 or 2, comprising 0, 1 or 2 point mutations in each of the CDR sequences which are the

CDR1-H1 sequence selected from the group consisting of SEQ ID NO 1, SEQ ID NO 7, SEQ ID NO 13, SEQ ID NO 19 and SEQ ID NO 26, and

CDR2-H1 sequence selected from the group consisting of SEQ ID NO 2, SEQ ID NO 8, SEQ ID NO 14, SEQ ID NO 20 and SEQ ID NO 27, and

CDR3-H1 sequence selected from the group consisting of SEQ ID NO 3, SEQ ID NO 9, SEQ ID NO 15 and SEQ ID NO 21, and

CDR1-L1 sequence selected from the group consisting of SEQ ID N04, SEQ ID NO 10, SEQ ID NO 16, SEQ ID NO 22 and SEQ ID NO 28, and

CDR2-L1 sequence selected from the group consisting of SEQ ID NO 5, SEQ ID NO 11, SEQ ID NO 17, SEQ ID NO 23 and SEQ ID NO 25, and

CDR3-L1 sequence selected from the group consisting of SEQ ID NO 6, SEQ ID NO 12, SEQ ID NO 18 and SEQ ID NO 24.

4. The anti-oxMIF/anti-CD3 antibody according to any one of items 1 to 3, wherein the binding site specifically recognizing CD3 comprises

(a) a heavy chain variable region comprising

a CDR1-H2 sequence which has at least 70% sequence identity to any of the sequences selected from the group consisting of SEQ ID NO 77, SEQ ID NO 86 and SEQ ID NO 92, and

a CDR2-H2 which has at least 70% sequence identity to any of the sequences selected from the group consisting of SEQ ID NO 78, SEQ ID NO 87, and SEQ ID NO 93, and

a CDR3-H2 which has at least 70% sequence identity to any of the sequences selected from the group consisting of SEQ ID NO 79, SEQ ID NO 88, SEQ ID NO 94, and SEQ ID NO 149, and

(b) a light chain comprising

a CDR1-L2 which has at least 70% sequence identity to any of the sequences selected from the group consisting of SEQ ID NO 80, SEQ ID NO 83, SEQ ID NO 89 and SEQ ID NO 95, and

a CDR2-L2 which has at least 70% sequence identity to any of the sequences selected from the group consisting of SEQ ID NO 81, SEQ ID NO 84, SEQ ID NO 90 and SEQ ID NO 96, and

a CDR3-L2 which has at least 70% sequence identity to any of the sequences selected from the group consisting of SEQ ID NO 82, SEQ ID NO 85, SEQ ID NO 91, SEQ ID NO 97 and SEQ ID NO 151.

5. The anti-oxMIF/anti-CD3 antibody according to any one of items 1 to 4, comprising 0, 1, or 2 point mutations in each of the CDR sequences which are the

CDR1-H2 sequence from the group consisting of SEQ ID NO 77, SEQ ID NO 86 and SEQ ID NO 92, and

CDR2-H2 sequence from the group consisting of SEQ ID NO 78, SEQ ID NO 87, and SEQ ID NO 93, and

CDR3-H2 sequence from the group consisting of SEQ ID NO 79, SEQ ID NO 88, SEQ ID NO 94, and SEQ ID NO 149, and

CDR1-L2 sequence from the group consisting of SEQ ID NO 80, SEQ ID NO 83, SEQ ID NO 89 and SEQ ID NO 95, and

CDR2-L2 sequence from the group consisting of SEQ ID NO 81, SEQ ID NO 84, SEQ ID NO 90 and SEQ ID NO 96, and

CDR3-L2 sequence from the group consisting of SEQ ID NO 82, SEQ ID NO 85, SEQ ID NO 91, SEQ ID NO 97, and SEQ ID NO 151.

6. The anti-oxMIF/anti-CD3 antibody according to any one of items 1 to 5, comprising the sequences SEQ ID NO 7, SEQ ID NO 8, SEQ ID NO 9, SEQ ID NO 10, SEQ ID NO 11, SEQ ID NO 12, SEQ ID NO 77, SEQ ID NO 78, SEQ ID NO 149, SEQ ID NO 83, SEQ ID NO 84, and SEQ ID NO 151.

7. The anti-oxMIF/anti-CD3 antibody according to any one of items 1 to 6, wherein the binding site specifically recognizing oxMIF comprises a heavy chain variable region having at least 70%, preferably at least 80%, preferably at least 90%, more preferably at least 95% sequence identity to the amino acid sequence of SEQ ID NO 172, and a light chain variable region having at least 70%, preferably at least 80%, preferably at least 90%, more preferably at least 95% sequence identity to the amino acid sequence of SEQ ID NO 134.

8. The anti-oxMIF/anti-CD3 antibody according to any one of items 1 to 6, wherein the binding site specifically recognizing CD3 comprises a heavy chain variable region having at least 70%, preferably at least 80%, preferably at least 90%, more preferably at least 95% sequence identity to the amino acid sequence of SEQ ID NO 135 and a light chain variable region having at least 70%, preferably at least 80%, preferably at least 90%, more preferably at least 95% sequence identity to the amino acid sequence of SEQ ID NO 136.

9. The anti-oxMIF/anti-CD3 antibody according to any one of items 1 to 8, wherein the at least one binding site is an antibody selected from the group consisting of scFv, (scFv)2, scFvFc, Fab, Fab′, and F(ab′)2, fusion proteins of two single chain antibodies of different species (BiTE), minibody, TandAb, DutaMab, and CrossMab.

10. The anti-oxMIF/anti-CD3 antibody according to any one of items 1 to 9, wherein the antibody comprises at least one antibody domain which is of human origin, or a chimeric, or humanized antibody domain of mammalian origin other than human, preferably of humanized, murine or camelid origin.

11. The anti-oxMIF/anti-CD3 antibody according to any one of items 1 to 10, comprising a monovalent, a bivalent, or a tetravalent binding site specifically binding oxMIF and a monovalent, a bivalent, or a tetravalent binding site specifically binding CD3.

12. The anti-oxMIF/anti-CD3 antibody according to any one of items 1 to 11, wherein the antibody is a bispecific antibody, specifically selected from the group consisting of bispecific IgG, IgG appended with a CD3 binding site, IgG appended with an oxMIF binding site, BsAb fragments, bispecific fusion proteins and BsAb conjugates.

13. A pharmaceutical composition comprising the anti-oxMIF/anti-CD3 antibody of items 1 to 12 and a pharmaceutically acceptable carrier or excipient.

14. The anti-oxMIF/anti-CD3 antibody according to any one of items 1 to 12 or the pharmaceutical composition of claim 13 for use in the treatment of cancer, specifically in the treatment of colorectal cancer, ovarian cancer, pancreas cancer, lung cancer.

15. The anti-oxMIF/anti-CD3 antibody according to any one of items 1 to 12 for use as a medicament.

16. A method for the treatment of a cancer comprising administering a therapeutically effective amount of a pharmaceutical composition according to item 13 to a subject in need thereof.

17. Isolated nucleic acid molecule(s) encoding an anti-oxMIF/anti-CD3 antibody according to any one of items 1 to 12.

18. An expression vector comprising nucleic acid molecule(s) of item 17.

19. A host cell comprising a vector according to item 18.

20. A method of producing the anti-oxMIF/anti-CD3 antibody according to any one of items 1 to 12, comprising expressing a nucleic acid encoding the antibody in a host cell.

21. An in vitro method of detecting cellular expression of oxMIF, the method comprising: contacting a biological sample comprising a human cell to be tested with an anti-oxMIF/anti-CD3 antibody according to any one of items 1 to 12; and

detecting binding of said antibody;

wherein the binding of said antibody indicates the presence of oxMIF on the cell, to thereby detect whether the cell expresses oxMIF.

22. The in vitro method of item 21, wherein the biological sample comprises intact human cells, tissues, biopsy probes, or a membrane fraction of a cell of interest.

23. The in vitro method of item 21 or 22, wherein the anti-oxMIF/anti-CD3 antibody is labeled with a detectable label selected from the group consisting of a radioisotope, a fluorescent label, a chemiluminescent label, an enzyme label, and a bioluminescent label.

24. The anti-oxMIF/anti-CD3 antibody of items 1 to 12 for use in diagnosing a cancer expressing oxMIF in a subject, wherein said antibody is conjugated to a detectable label.

The foregoing description will be more fully understood with reference to the following examples. Such examples are, however, merely representative of methods of practicing one or more embodiments of the present invention and should not be read as limiting the scope of invention.

### EXAMPLES

### Example 1

### Biochemical Characterization of Bispecific Antibodies

The anti-oxMIF/anti-CD3 antibodies are tested as described below to ensure quality and functionality.

1) Identity: Method: by Electrospray ionization MS (ESI-MS)

2) Molecular integrity: Method: SEC multi-angle light scattering (SEC MALS)

3) Purity: Method: SDS PAGE

4) Binding and affinity: Methods: ELISA, Biacore, FACS as described below

ELISA according to Thiele M. et al., 2015, J Immunol 2015; 195:2343-2352: For determination of oxMIF specificity, anti-oxMIF/anti-CD3 antibodies are coated into microplates and incubated with recombinant MIF (control), oxMIF, or oxMIF reduced with DTT (control). Captured MIF or oxMIF is detected with rabbit anti-MIF Abs and a goat anti-rabbit-IgG-HRP conjugate. Plates are stained with 3,3′,5,5′-Tetramethylbenzidine. For determination of CD3 specificity, anti-oxMIF/anti-CD3 antibodies are coated into microplates and incubated with recombinant Human CD3 epsilon protein. Captured CD3 is detected with rabbit anti-CD3 Abs and a goat anti-rabbit-IgG-HRP conjugate. Plates are stained with 3,3′,5,5′-Tetramethylbenzidine.

SPR (Biacore) according to Hoellriegl et al., Eur J Pharmacol. 2018 February 5; 820:206-216: Binding affinities and kinetic constants of anti-oxMIF/CD3 bispecific antibodies are determined by surface plasmon resonance using either an antibody-capture format (anti-oxMIF/CD3 bispecific abs captured on sensor chip) or an antigen-capture format (recombinant MIF or recombinant CD3 (epsilon, delta or gamma chain) captured on a sensor chip). Measurements are conducted on a T200 Biacore instrument.

Specifically, anti-oxMIF/anti-CD3 antibody or a non-binding control antibody is immobilized to Biacore CM5 optical sensor chips (GE Healthcare, Piscataway, N.J.) using standard amine coupling conditions. Recombinant MIF is diluted in HBS-EP buffer (GE Healthcare) to concentrations of 50, 75, 100, or 150 nM in the presence of 0.2% Proclin300 (active component 5-chloro-2-methyl-4-isothiazolin-3-one; Sigma) to transform MIF into an oxMIF surrogate (Thiele M. et al., 2015, J Immunol 2015; 195:2343-2352). Proclin300 treated MIF is applied to immobilized anti-oxMIF/anti-CD3 antibody and affinity measured with a Biacore™ 3000 Instrument (GE Healthcare). The kinetics of the concentration series are analyzed by local simultaneous association/dissociation fitting of each binding curve to the iterative Langmuir 1:1 interaction model with mass transfer compensation provided by the BiaEvaluation software (GE Healthcare).

FACS: oxMIF positive cancer cells (e.g. PC3 or A2780) are incubated with anti-oxMIF/anti-CD3 bispecific abs or controls. Unlabeled Abs are detected by R-PE-labeled goat anti-human IgG Ab (from Sigma). Data are acquired on a FACS Canto II (BD Biosciences).

### Example 2

### In Vitro Efficacy of Bispecific Antibodies

The anti-oxMIF/anti-CD3 antibodies are tested for in vitro activity in a T Cell-Mediated Tumor Cell Lysis Assay. EC50 values are determined.

The assay format is as follows: Primary t cells are isolated from different donors and co-cultured with calcein loaded tumor cells at specific effector cell:target cell (E:T) ratios. The bispecific anti-oxMIF/CD3 antibodies and respective controls are added to the co-culture and tumor cell lysis is monitored via calcein release.

Biodistribution and PK Study

Biodistribution and pharmacokinetics (PK) of the anti-oxMIF/anti-CD3 antibodies are determined by PET-imaging. The bispecific anti-oxMIF/anti-CD3 antibodies are labelled and pharmacokinetics of the proteins in the tumor, circulation and major organs are determined in SCID mice bearing a subcutaneous SKOV-3 tumor or another appropriate cell line.

Exploratory PD Study

1) Xenograft NOD/SCID SKOV-3 model: A dose response curve of the anti-oxMIF/anti-CD3 bispecific antibodies is determined in a NOD/SCID SKOV-3 xenograft mouse model for ovarian cancer applying human lymphocytes (Xing, J., et al., Translational Oncology (2017) 10, 780-785)

Briefly, fresh cultured SKOV-3 cells (1×10⁶) are mixed with fresh isolated human PBMCs (5×10⁶) in 200-μl volume and subcutaneously co-implanted into the right flank of 5-week-old male NOD/SCID mice. Two hours after tumor cell injection, mice are treated with anti-oxMIF/anti-CD3 antibodies every 3 days by intraperitoneal injection. The anti-oxMIF/anti-CD3 bispecific antibodies are applied in 6 doses, the respective control bispecific antibodies in the highest dose. Mice are weighed and tumor growth is measured twice a week using calipers. Tumor volume is calculated as ½(length×width²).

As an alternative, PD of anti-oxMIF/anti-CD3 antibodies is monitored by bioluminescence. Briefly, thirty 5-weeks old NSG mice (The Jackson Laboratory) are each given 1×10⁶ IGROV1-ffluc intraperitoneally (i.p.) on day 0. On day 2, the animals are i.p. injected with 150 mg/kg D-luciferin (15 mg/mL stock solution; Biosynth) and divided into 5 groups of 6 animals each by average bioluminescence. On day 6, each animal (except the no treatment cohort) is i.p. injected with 1×10⁷ primary T cells expanded from healthy donor PBMC, and 1 h later, with anti-oxMIF/CD3 antibodies in 4 different doses or PBS alone. This is repeated for a total of 10 daily (day 6 to 15) i.p. injections. Every 3-4 days, tumor growth is monitored by bioluminescent imaging 5 min after i.p. injections with 150 mg/kg D-luciferin. The weight of the mice is measured every 1-4 days.

2) Primary ovarian human xenograft model: The anti-oxMIF/anti-CD3 bispecific antibodies are tested essentially as described in Schleret B. et al., Cancer Res 2005; 65(7): 2882-9.

In brief, following surgical resection of peritoneal metastasis of histologically proven ovarian cancer patients, primary tumor specimens are cut into 50 to 100 mm3 cubes and s.c. implanted into NOD/SCID mice. Animals are i.v. treated with anti-oxMIF/anti-CD3 bispecific antibody formats or control antibody. The anti-oxMIF/anti-CD3 bispecific antibodies are applied in 3 doses, the respective control in the highest dose. Tumor sizes are measured twice a week with a caliper in two perpendicular dimensions and tumor volumes calculated according to tumor volume=[(width²×length)/2].

As an alternative: 1×10⁶ human PBMCs isolated from heparinized fresh whole blood of a healthy donor are mixed with 5×10⁵ primary tumor-initiating cells (TICs) in a final volume of 200 μl. The PBMC effector/target cell mixture (E:T of 2:1) is s.c. injected into the right flank of each NOD/SCID mouse. The mice are intravenously treated with anti-oxMIF/CD3 antibodies or PBS control vehicle starting 2 h after inoculation with 3 different doses.

For elimination of established tumors in NOD/SCID mice by treatment with anti-oxMIF/CD3 antibodies, mixtures of 5×10⁶ TICs and 1×10⁷ human PBMCs are inoculated into 5 NOD/SCID mice per group to allow solid tumor formation. After tumor establishment at day 4, mice are treated i.v. for 14 days with three different doses of anti-oxMIF/CD3 antibodies, or with vehicle control in presence of PBMCs.

### Example 3

Overview on antibody formats used in the examples.

C0036 (Anti-oxMIF Fab fused to anti-CD3 scFv (HC);
format: Fab-scFv):
Polypeptide 1:
(SEQ ID NO 153)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKRVEPKSCGGGGSGGGGSGGGGSQVQLVQSGAEVKKPGASVK
VSCKASGYTFTRYTMHWVRQAPGQGLEWMGYINPSRGYTNYNQKFKDRVTL
TTDKSSSTAYMELSSLRSEDTAVYYCARYYDDHYSLDYWGQGTLVTVSSGG
SGGSGGSGGSGGSDIQMTQSPSSLSASVGDRVTITCSASSSVSYMNWYQQK
PGKAPKRLIYDTSKLASGVPSRFSGSGSGTDFTLTISSLQPEDFATYYCQQ
WSSNPFTFGQGTKLEIKAAAEQKLISEEDLAAHHHHHH.
Polypeptide 2:
(SEQ ID NO 154)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.
C0037 (Anti-oxMIF Fab fused to two anti-CD3 scFv.
format: Fab-(scFv)2):
Polypeptide 1:
(SEQ ID NO 155)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKRVEPKSCGGGGSGGGGSGGGGSQVQLVQSGAEVKKPGASVK
VSCKASGYTFTRYTMHWVRQAPGQGLEWMGYINPSRGYTNYNQKFKDRVTL
TTDKSSSTAYMELSSLRSEDTAVYYCARYYDDHYSLDYWGQGTLVTVSSGG
SGGSGGSGGSGGSDIQMTQSPSSLSASVGDRVTITCSASSSVSYMNWYQQK
PGKAPKRLIYDTSKLASGVPSRFSGSGSGTDFTLTISSLQPEDFATYYCQQ
WSSNPFTFGQGTKLEIKAAAEQKLISEEDLAAHHHHHH.
Polypeptide 2:
(SEQ ID NO 156)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGECGGGSGGGSGGGSQVQLVQSGAEVKKPGASVKVSCKASGYTF
TRYTMHWVRQAPGQGLEWMGYINPSRGYTNYNQKFKDRVTLTTDKSSSTAY
MELSSLRSEDTAVYYCARYYDDHYSLDYWGQGTLVTVSSGGSGGSGGSGGS
GGSDIQMTQSPSSLSASVGDRVTITCSASSSVSYMNWYQQKPGKAPKRLIY
DTSKLASGVPSRFSGSGSGTDFTLTISSLQPEDFATYYCQQWSSNPFTFGQ
GTKLEIKASAWSHPQFEK.
C0038 (Anti-oxMIF Fab and anti-CD3 scFy fused to
Fc, Fab-scFv-Fc):
Polypeptide 1:
(SEQ ID NO 157)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.
Polypeptide 2:
(SEQ ID NO 158)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPCRD
ELTKNQVSLWCLVKGFYPSDIAVEWESNGQPENNYKTTPPVLDSDGSFFLY
SKLTVDKSRWQQGNVFSCSVMHEALHNHYTQKSLSLSPGK.
Polypeptide 3:
(SEQ ID NO 159)
QVQLVQSGAEVKKPGASVKVSCKASGYTFTRYTMHWVRQAPGQGLEWMGYI
NPSRGYTNYNQKFKDRVTLTTDKSSSTAYMELSSLRSEDTAVYYCARYYDD
HYSLDYWGQGTLVTVSSGGSGGSGGSGGSGGSDIQMTQSPSSLSASVGDRV
TITCSASSSVSYMNWYQQKPGKAPKRLIYDTSKLASGVPSRFSGSGSGTDF
TLTISSLQPEDFATYYCQQWSSNPFTFGQGTKLEIKGGGGSDKTHTCPPCP
APELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPI
EKTISKAKGQPREPQVCTLPPSRDELTKNQVSLSCAVKGFYPSDIAVEWES
NGQPENNYKTTPPVLDSDGSFFLVSKLTVDKSRWQQGNVFSCSVMHEALHN
HYTQKSLSLSPGK.
C0039 (Full anti-oxMIF IgG with anti-CD3 scFy fused
to light chain, IgG(Ic)-scFv):
Polypeptide 1:
(SEQ ID NO 160)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLY
SKLTVDKSRWQQGNVFSCSVMHEALHNHYTQKSLSLSPG.
Polypeptide 2:
(SEQ ID NO 161)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGECGGGGSGGGGSGGGGSQVQLVQSGAEVKKPGASVKVSCKASG
YTFTRYTMHWVRQAPGQGLEWMGYINPSRGYTNYNQKFKDRVTLTTDKSSS
TAYMELSSLRSEDTAVYYCARYYDDHYSLDYWGQGTLVTVSSGGSGGSGGS
GGSGGSDIQMTQSPSSLSASVGDRVTITCSASSSVSYMNWYQQKPGKAPKR
LIYDTSKLASGVPSRFSGSGSGTDFTLTISSLQPEDFATYYCQQWSSNPFT
FGQGTKLEIK.
C0006 (Anti-oxMIF/CD3 Crossmab (CH1-CL), Crossmab)
Polypeptide 1:
(SEQ ID NO 162)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.
Polypeptide 2:
(SEQ ID NO 163)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPCRD
ELTKNQVSLWCLVKGFYPSDIAVEWESNGQPENNYKTTPPVLDSDGSFFLY
SKLTVDKSRWQQGNVFSCSVMHEALHNHYTQKSLSLSPGK.
Polypeptide 3:
(SEQ ID NO 164)
QVQLVQSGAEVKKPGASVKVSCKASGYTFTRYTMHWVRQAPGQGLEWMGYI
NPSRGYTNYNQKFKDRVTLTTDKSSSTAYMELSSLRSEDTAVYYCARYYDD
HYSLDYWGQGTLVTVSSASVAAPSVFIFPPSDEQLKSGTASVVCLLNNFYP
REAKVQWKVDNALQSGNSQESVTEQDSKDSTYSLSSTLTLSKADYEKHKVY
ACEVTHQGLSSPVTKSFNRGECDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCTL
PPSRDELTKNQVSLSCAVKGFYPSDIAVEWESNGQPENNYKTTPPVLDSDG
SFFLVSKLTVDKSRWQQGNVFSCSVMHEALHNHYTQKSLSLSPGK.
Polypeptide 4:
(SEQ ID NO 165)
DIQMTQSPSSLSASVGDRVTITCSASSSVSYMNWYQQKPGKAPKRLIYDTS
KLASGVPSRFSGSGSGTDFTLTISSLQPEDFATYYCQQWSSNPFTFGQGTK
LEIKSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDK
KVEPKSC.
C0007 (Full anti-oxMIF IgG with anti-CD3 scFv fused
to heavy chain, v IgG1-scFv fusion)
Polypeptide 1:
(SEQ ID NO 166)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLY
SKLTVDKSRWQQGNVFSCSVMHEALHNHYTQKSLSLSPGKGGGGSGGGGSG
GGGSQVQLVQSGAEVKKPGASVKVSCKASGYTFTRYTMHWVRQAPGQGLEW
MGYINPSRGYTNYNQKFKDRVTLTTDKSSSTAYMELSSLRSEDTAVYYCAR
YYDDHYSLDYWGQGTLVTVSSGGSGGSGGSGGSGGSDIQMTQSPSSLSASV
GDRVTITCSASSSVSYMNWYQQKPGKAPKRLIYDTSKLASGVPSRFSGSGS
GTDFTLTISSLQPEDFATYYCQQWSSNPFTFGQGTKLEIK.
Polypeptide 2:
(SEQ ID NO 167)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.
C0032 (Anti-oxMIF-scFv fused to an anti-CD3-scFv,
BiTE)
Polypeptide 1
(SEQ ID NO 168)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKGGGGSGGGGSGGGGSEVQLLESGGGLVQPGGSLRLSCAASGFTFSI
YSMNWVRQAPGKGLEWVSSIGSSGGTTYYADSVKGRFTISRDNSKNTLYLQ
MNSLRAEDTAVYYCAGSQWLYGMDVWGQGTTVTVSSGGGGSQVQLVQSGAE
VKKPGASVKVSCKASGYTFTRYTMHWVRQAPGQGLEWMGYINPSRGYTNYN
QKFKDRVTLTTDKSSSTAYMELSSLRSEDTAVYYCARYYDDHYSLDYWGQG
TLVTVSSGGSGGSGGSGGSGGSDIQMTQSPSSLSASVGDRVTITCSASSSV
SYMNWYQQKPGKAPKRLIYDTSKLASGVPSRFSGSGSGTDFTLTISSLQPE
DFATYYCQQWSSNPFTFGQGTKLEIKAAAEQKLISEEDLSAWSHPQFEK
C0033 (Variable domains of anti-oxMIF and anti-CD3-
fused in a row (VL1-VH2-VL2-VH1, TandAb)
Polypeptide 1
(SEQ ID NO 169)
DIQMTQSPSSLSASVGDRVTITCSASSSVSYMNWYQQKPGKAPKRLIYDTS
KLASGVPSRFSGSGSGTDFTLTISSLQPEDFATYYCQQWSSNPFTFGQGTK
LEIKGGSGGSEVQLLESGGGLVQPGGSLRLSCAASGFTFSIYSMNWVRQAP
GKGLEWVSSIGSSGGTTYYADSVKGRFTISRDNSKNTLYLQMNSLRAEDTA
VYYCAGSQWLYGMDVWGQGTTVTVSSGGSGGSDIQMTQSPSSLSASVGDRV
TITCRSSQRIMTYLNWYQQKPGKAPKLLIFVASHSQSGVPSRFRGSGSETD
FTLTISGLQPEDSATYYCQQSFWTPLTFGGGTKVEIKGGSGGSQVQLVQSG
AEVKKPGASVKVSCKASGYTFTRYTMHWVRQAPGQGLEWMGYINPSRGYTN
YNQKFKDRVTLTTDKSSSTAYMELSSLRSEDTAVYYCARYYDDHYSLDYWG
QGTLVTVSSAAAEQKLISEEDLSAWSHPQFEK.
C0008 (full anti-oxMIF IgG, IgG1)
Polypeptide 1
(SEQ ID NO 170)
EVQLLESGGGLVQPGGSLRLSCAASGFTFSIYSMNWVRQAPGKGLEWVSSI
GSSGGTTYYADSVKGRFTISRDNSKNTLYLQMNSLRAEDTAVYYCAGSQWL
YGMDVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVN
HKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLY
SKLTVDKSRWQQGNVFSCSVMHEALHNHYTQKSLSLSPG.
Polypeptide 2
(SEQ ID NO 171)
DIQMTQSPSSLSASVGDRVTITCRSSQRIMTYLNWYQQKPGKAPKLLIFVA
SHSQSGVPSRFRGSGSETDFTLTISGLQPEDSATYYCQQSFWTPLTFGGGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

### Example 4

### oxMIF ELISA

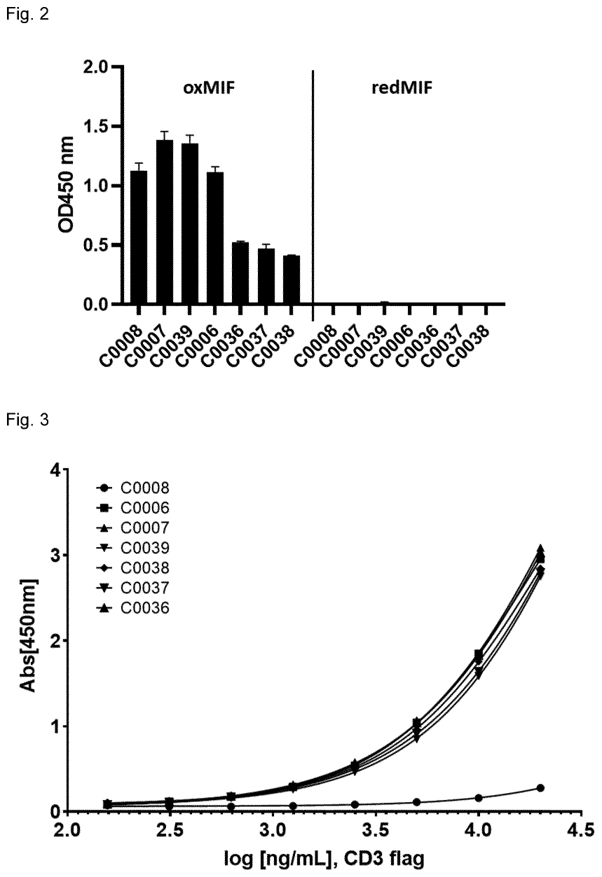

For determination of oxMIF specificity, bispecific antibodies were immobilized into microwell plates at 2 μg/ml in PBS. After blocking with 2% BSA/TBST, the wells were incubated with 500 ng/ml of either redMIF or the oxMIF surrogate TNB-MIF (MIF treated with DTNB according to Schinagl et al., Biochemistry, 2018, 57 (9), pp 1523-1532). Captured TNB-MIF was detected with a polyclonal rabbit anti-MIF goat anti-rabbit IgG-HRP conjugate. Plates were stained with tetramethylbenzidine and chromogenic reaction was stopped with H₂SO₄. OD was measured at 450 nM. oxMIF specificity of bispecific antibodies is shown in FIG. 2 (C0008 represents the monospecific anti-oxMIF antibody as a positive control).

### Example 5

### oxMIF-CD3 Bridging ELISA

Recombinant human MIF was immobilized into microwell plates at 1 μg/ml in PBS (transforming MIF to oxMIF according to Thiele M. et al., 2015, J Immunol 2015; 195:2343-2352). After blocking bispecific antibodies were added to the plates at a concentration of 4 μg/ml. A dilution series of a FLAG-taggedCD3-ϵ-δ-Fc fusion protein was added and bound CD3 was detected using a monoclonal mouse anti-FLAG tag-HRP. OD was measured at 450 nM.

Simultaneously binding of bispecific antibodies to oxMIF and CD3 is shown in FIG. 3. C0008 represents the monospecific anti-oxMIF antibody as a negative control.

### Example 6

### Binding of Bispecific Antibodies to Native CD3 on T Cells

CD3 positive Jurkat T-cells, which express functional CD3 (CD3+) were incubated with bispecific antibodies, C0008 (anti-oxMIF monospecific control antibody), a non-specific isotype control antibody (Isotype) at a concentration of 70 nM or without antibody (secondary ab only). Bound antibodies were detected by a goat anti-human IgG (H+L) Alexa-Fluor 488 conjugate (secondary antibody). 7AAD was used to label dead cells and samples were analysed by FACS.

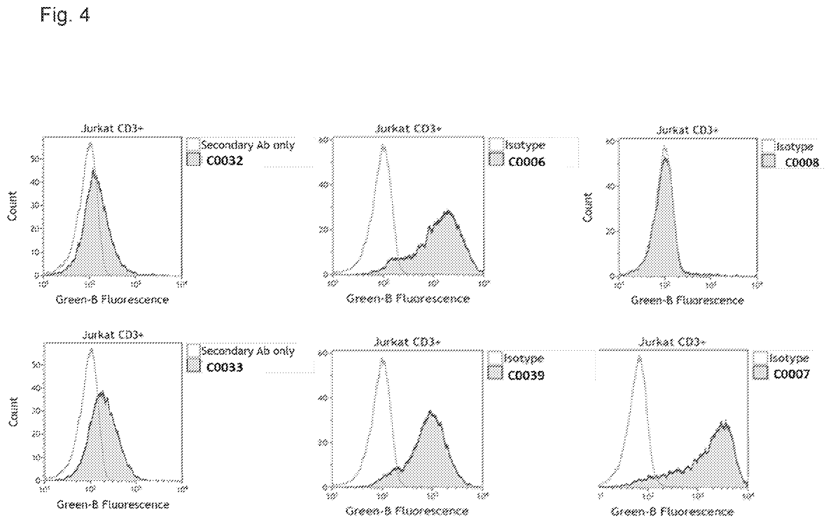

Detection of native CD3 on viable Jurkat T cells with anti-oxMIF/CD3 bispecific antibodies) is shown in FIG. 4.

### Example 7

### Binding to oxMIF (ELISA)

Recombinant human MIF (1 μg/ml) diluted in PBS was immobilized into microwell plates (transforming MIF to oxMIF according to Thiele M. et al., 2015, J Immunol 2015; 195:2343-2352). After blocking, the bispecific antibodies were added to the plates at different concentrations. Bound bispecific antibodies were detected using protein L-HRP conjugate. Plates were developed by adding TMB and chromogenic reaction was stopped with H₂SO₄. OD was measured at 450 nM.

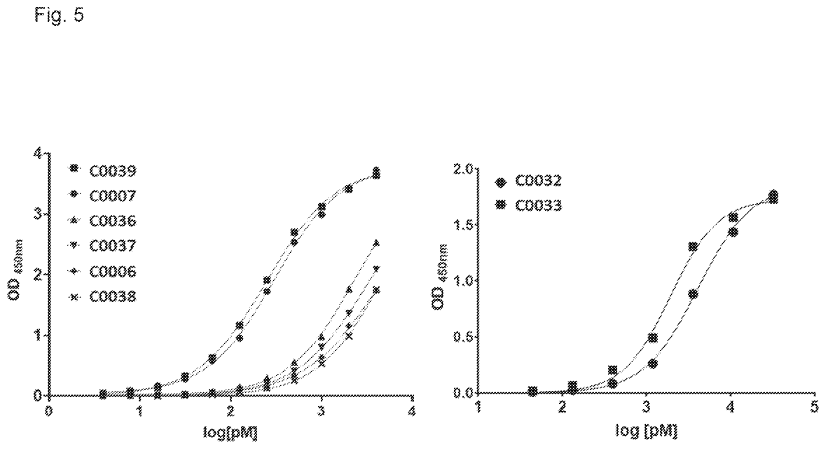

The curves of anti-oxMIF/CD3 bispecific antibody binding towards immobilized MIF (oxMIF) are shown in FIG. 5. EC50 values of the binding curves, which reflect rough KD estimates, were calculated by 4-parameter fit and are shown in Table 5.

TABLE 5
EC50 values of bispecific antibodies (ELISA):

| Entity | EC₅₀ (nM) |
|---|---|
| C0039 | 0.3 |
| C0007 | 0.3 |
| C0036 | 2.3 |
| C0037 | 3.4 |
| C0006 | 4.6 |
| C0038 | 4.6 |
| C0032 | 4.0 |
| C0033 | 1.9 |

### Example 8

### Affinity of Bispecific Antibodies (SPR)

The affinity of the antibodies was determined using a Biacore™ T200 device. HuMIF was immobilized onto CM5 sensor chips and anti-oxMIF bispecific entities were injected at different concentrations in running buffer (HBS-EP plus 0.1% BSA) to generate single cycle kinetic profiles. Affinity constants were calculated according to the 1:1 Langmuir model and are shown in Table 6.

TABLE 6
Affinity constants (KD) of bispecific antibodies

| | KD (M) |
|---|---|
| C0006 | 7.2E−09 |
| C0007 | 6.1E−10 |
| C0036 | 2.3E−09 |
| C0037 | 5.9E−09 |
| C0039 | 1.0E−09 |
| C0038 | 9.4E−09 |
| C0032 | 4.3E−09 |
| C0033 | 4.8E−09 |

### Example 9

Binding of bispecific antibodies to native oxMIF on the surface of ovarian cancer cells.

A2780 ovarian cancer cells were incubated with bispecific antibodies, C0008 (anti-oxMIF monospecific control antibody), a non-specific isotype control antibody (Isotpye) at a concentration of 70 nM in 5% BSA/PBS or in 5% BSA/PBS without antibody (Sec. only). Bound bispecific molecules were detected with a goat anti-human IgG (H+L) AlexaFluor 488 conjugate (secondary antibody). 7AAD was used to label dead cells and samples were analysed by FACS.

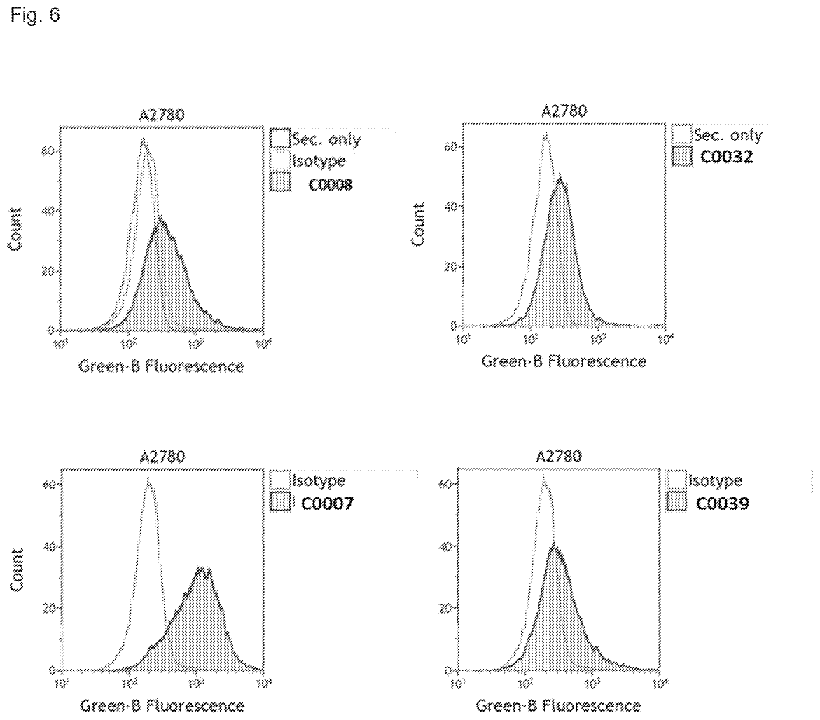

Binding of anti-oxMIF/CD3 bispecific antibodies to native oxMIF on the cell surface of A2780 ovarian cancer cells is shown in FIG. 6.

### Example 10

### Activation of T Cells by Anti-oxMIF/CD3 BiTE

The T Cell Activation Bioassay was done according to the Promega technical manual for product J1621 by using genetically engineered Jurkat T cells (effector cells) that express a luciferase reporter driven by a NFAT-response element. The assay was done either in the presence or in the absence of A278 ovarian cancer cells (Target cells) at an Effector:Target (E:T) cell ratio of 2.5:1

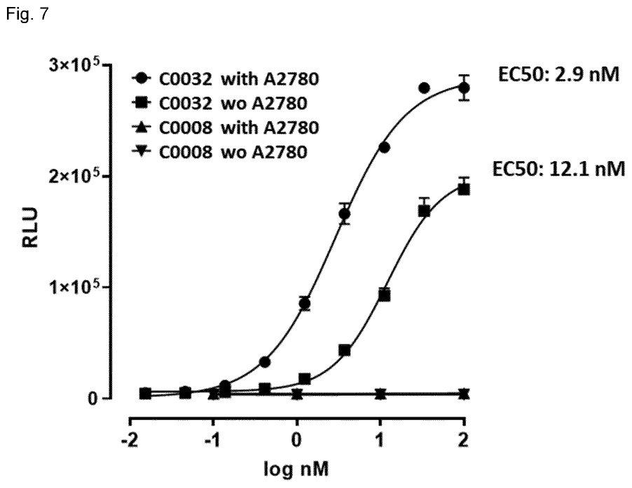

Activation of T cells by anti-oxMIF/CD3 bispecific entity C0032 vs anti-oxMIF monospecific control antibody C0008 in the presence of A2780 ovarian cancer cells is shown in FIG. 7.

### Example 11

### PBMC Mediated Tumor Cell Killing

A2780 ovarian and A549 lung cancer cells were seeded in 96 well V-bottom plates. PBMCs were isolated from blood of healthy, human donors. Serial dilutions of anti-oxMIF/CD3 Crossmab were added to the tumor cells together with PBMCs and incubated for 2.5 h (Effector-to-target cell ratio: 10:1). Cell culture supernatants were transferred into new plates and activity of released proteases was analyzed by using the Promega Cytotox-Glo™ assay. The remaining cells were lysed, and the protease activity of the lysate was analysed by using the Promega Cytotox-Glo™ assay

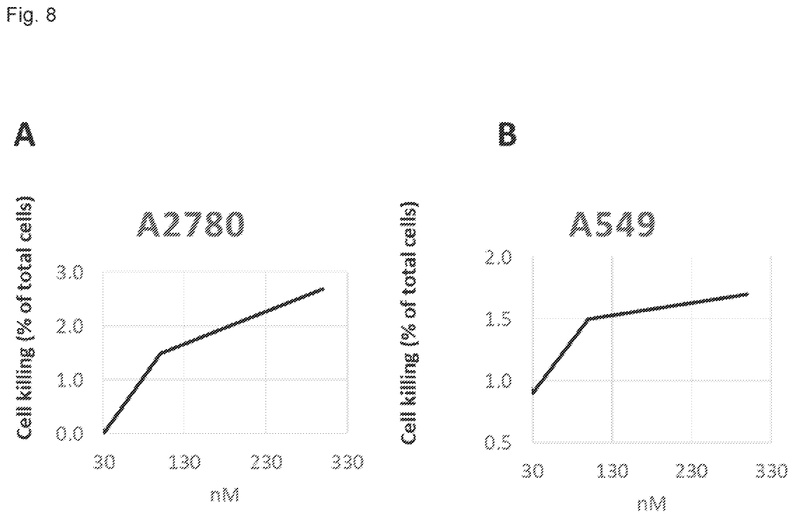

PBMC mediated tumor cell killing of A2780 ovarian cancer cells (A) and A549 lung cancer cells (B) in the presence of C0006 is shown in FIG. 8. Ratio of protease activity of the supernatant/activity of lysate×100=% Cell killing.

1. An anti-oxMIF/anti-CD3 antibody comprising at least one binding site specifically recognizing oxMIF and at least one binding site specifically recognizing CD3.

2. The anti-oxMIF/anti-CD3 antibody of claim 1, wherein the binding site specifically recognizing oxMIF comprises
(a) a heavy chain variable region comprising
a CDR1-H1 sequence which has at least 70% sequence identity to any of the sequences selected from the group consisting of SEQ ID NO: 1, SEQ ID NO: 7, SEQ ID NO: 13, SEQ ID NO: 19 and SEQ ID NO: 26, and
a CDR2-H1 sequence which has at least 70% sequence identity to any of the sequences selected from the group consisting of SEQ ID NO: 2, SEQ ID NO: 8, SEQ ID NO: 14, SEQ ID NO: 20 and SEQ ID NO: 27, and
a CDR3-H1 sequence which has at least 70% sequence identity to any of the sequences selected from the group consisting of SEQ ID NO: 3, SEQ ID NO: 9, SEQ ID NO: 15 and SEQ ID NO: 21, and
(b) a light chain variable region comprising
a CDR1-L1 sequence which has at least 70% sequence identity to any of the sequences selected from the group consisting of SEQ ID NO: 4, SEQ ID NO: 10, SEQ ID NO: 16, SEQ ID NO: 22 and SEQ ID NO: 28, and
a CDR2-L1 sequence which has at least 70% sequence identity to any of the sequences selected from the group consisting of SEQ ID NO: 5, SEQ ID NO: 11, SEQ ID NO: 17, SEQ ID NO: 23 and SEQ ID NO: 25, and
a CDR3-L1 sequence which has at least 70% sequence identity to any of the sequences selected from the group consisting of SEQ ID NO: 6, SEQ ID NO: 12, SEQ ID NO: 18 and SEQ ID NO: 24.

3. The anti-oxMIF/anti-CD3 antibody of claim 2, comprising 0, 1, or 2 point mutations in each CDR sequences, wherein the CDR sequences are
a CDR1-H1 sequence selected from the group consisting of SEQ ID NO: 1, SEQ ID NO: 7, SEQ ID NO: 13, SEQ ID NO: 19 and SEQ ID NO: 26, and
a CDR2-H1 sequence selected from the group consisting of SEQ ID NO: 2, SEQ ID NO: 8, SEQ ID NO: 14, SEQ ID NO: 20 and SEQ ID NO: 27, and
a CDR3-H1 sequence selected from the group consisting of SEQ ID NO: 3, SEQ ID NO: 9, SEQ ID NO: 15 and SEQ ID NO: 21, and
a CDR1-L1 sequence selected from the group consisting of SEQ ID NO: 4, SEQ ID NO: 10, SEQ ID NO: 16, SEQ ID NO: 22 and SEQ ID NO: 28, and
a CDR2-L1 sequence selected from the group consisting of SEQ ID NO: 5, SEQ ID NO: 11, SEQ ID NO: 17, SEQ ID NO: 23 and SEQ ID NO: 25, and
a CDR3-L1 sequence selected from the group consisting of SEQ ID NO: 6, SEQ ID NO: 12, SEQ ID NO: 18 and SEQ ID NO: 24.

4. The anti-oxMIF/anti-CD3 antibody according to claim 1, wherein the binding site specifically recognizing CD3 comprises
(a) a heavy chain variable region comprising
a CDR1-H2 sequence which has at least 70% sequence identity to any of the sequences selected from the group consisting of SEQ ID NO: 77, SEQ ID NO: 86 and SEQ ID NO: 92, and
a CDR2-H2 which has at least 70% sequence identity to any of the sequences selected from the group consisting of SEQ ID NO: 78, SEQ ID NO: 87, and SEQ ID NO: 93, and
a CDR3-H2 which has at least 70% sequence identity to any of the sequences selected from the group consisting of SEQ ID NO: 79, SEQ ID NO: 88, SEQ ID NO: 94, and SEQ ID NO: 149, and
(b) a light chain comprising
a CDR1-L2 which has at least 70% sequence identity to any of the sequences selected from the group consisting of SEQ ID NO: 80, SEQ ID NO: 83, SEQ ID NO: 89 and SEQ ID NO: 95, and
a CDR2-L2 which has at least 70% sequence identity to any of the sequences selected from the group consisting of SEQ ID NO: 81, SEQ ID NO: 84, SEQ ID NO: 90 and SEQ ID NO: 96, and
a CDR3-L2 which has at least 70% sequence identity to any of the sequences selected from the group consisting of SEQ ID NO: 82, SEQ ID NO: 85, SEQ ID NO: 91, SEQ ID NO: 97, and SEQ ID NO: 151.

5. The anti-oxMIF/anti-CD3 antibody according to claim 1 comprising 0, 1, or 2 point mutations in each CDR sequences, wherein the CDR sequences are
a CDR1-H2 sequence from the group consisting of SEQ ID NO: 77, SEQ ID NO: 86 and SEQ ID NO: 92, and
a CDR2-H2 sequence from the group consisting of SEQ ID NO: 78, SEQ ID NO: 87, and SEQ ID NO: 93, and
a CDR3-H2 sequence from the group consisting of SEQ ID NO: 79, SEQ ID NO: 88, SEQ ID NO: 94, and SEQ ID NO: 149, and
a CDR1-L2 sequence from the group consisting of SEQ ID NO: 80, SEQ ID NO: 83, SEQ ID NO: 89 and SEQ ID NO: 95, and
a CDR2-L2 sequence from the group consisting of SEQ ID NO: 81, SEQ ID NO: 84, SEQ ID NO: 90 and SEQ ID NO: 96, and
a CDR3-L2 sequence from the group consisting of SEQ ID NO: 82, SEQ ID NO: 85, SEQ ID NO: 91, SEQ ID NO: 97, and SEQ ID NO: 151.

6. The anti-oxMIF/anti-CD3 antibody according to claim 1, wherein the anti-oxMIF/anti-CD3 antibody comprises the sequences SEQ ID NO: 7, SEQ ID NO: 8, SEQ ID NO: 9, SEQ ID NO: 10, SEQ ID NO: 11, SEQ ID NO: 12, SEQ ID NO: 77, SEQ ID NO: 78, -SEQ ID NO: 149, SEQ ID NO: 83, SEQ ID NO :84, and SEQ ID NO: 151.

7. The anti-oxMIF/anti-CD3 antibody according to claim 1, wherein the binding site specifically recognizing oxMIF comprises a heavy chain variable region having at least 70%, or at least 80%, or at least 90%, or at least 95%, or at least 99.5% sequence identity to the amino acid sequence of SEQ ID NO: 172, and a light chain variable region having at least 70%, or at least 80%, or at least 90%, or at least 95% sequence, or at least 99.5% identity to the amino acid sequence of SEQ ID NO: 134.

8. The anti-oxMIF/anti-CD3 antibody according to claim 1, wherein the binding site specifically recognizing CD3 comprises a heavy chain variable region having at least 70%, or at least 80%, or at least 90%, or at least 95% sequence identity to the amino acid sequence of SEQ ID NO: 135 and a light chain variable region having at least 70%, or at least 80%, or at least 90%, or at least 95% sequence identity to the amino acid sequence of SEQ ID NO: 136.

9. The anti-oxMIF/anti-CD3 antibody according to claim 1, wherein the at least one binding site is an antibody selected from the group consisting of scFv, (scFv)2, scFvFc, Fab, Fab′, and F(ab′)2, Fab′-SH, Fab-scFv fusion, Fab-(scFv)2-fusion, Fab-scFv-Fc, fusion proteins of two single chain antibodies of different species (BiTE), minibody, TandAb, DutaMab, DART, and CrossMab.

10. The anti-oxMIF/anti-CD3 antibody according to claim 1, comprising a monovalent, a bivalent, or a tetravalent binding site specifically binding oxMIF and a monovalent, a bivalent or a tetravalent binding site specifically binding CD3.

11. The anti-oxMIF/anti-CD3 antibody of claim 1, wherein the antibody is combined with a pharmaceutically acceptable carrier or excipient to form a pharmaceutical composition.

12. A method of treating cancer, comprising the step of administering a therapeutically effective amount of the anti-oxMIF/anti-CD3 of claim 1 to a subject in need thereof,

13. (canceled)

14. A nucleic acid molecule encoding an anti-oxMIF/anti-CD3 antibody according to claim 1.

15. The nucleic acid molecule of claim 14, wherein the nucleic acid molecule is incorporated into an expression vector.

16. The method of claim 12, wherein the cancer being treated is selected from the group consisting of colorectal cancer, ovarian cancer, pancreas cancer, and lung cancer.
